(19) United States
(12) Patent Application Publication
(10) Pub. No.: US 2017/0233815 A1
(43) Pub. Date: Aug. 17, 2017

(54) HEALTHCARE DIAGNOSTIC

(71) Applicant: James Archibald TIMMONS, London (GB)

(72) Inventor: James Archibald TIMMONS, London (GB)

(21) Appl. No.: 15/503,619

(22) PCT Filed: Aug. 11, 2015
(86) PCT No.: PCT/GB2015/052314

§ 371 Date: Feb. 13, 2017

(30) Foreign Application Priority Data
Aug. 13, 2014 (GB) 1414317.6
May 28, 2015 (GB) 1509128.3

(51) Int. Cl. C12Q 1/68

(52) U.S. Cl. CPC C12Q 1/6883; C12Q 2600/158; C12Q 2600/118

(57) ABSTRACT

A health-ageing biomarker is provided which has utility in assessing the biological age of an individual. The biomarker has particular utility in the prediction of the likelihood of an individual developing an ageing-related disease, screening for anti-ageing drugs and to assist with the diagnosis of an ageing-related disease, or assessing the likelihood of an organ being successfully used or matched to a donor patient. Also presented are methods utilising the biomarker and methods of identifying such biomarkers.

### FIELD OF THE INVENTION

This invention relates to the use of genes, and gene expression, as a biomarker in the context of healthcare and medical diagnostics, and related medical tests and methods, in relation to the ageing of an individual and ageing-related diseases.

### BACKGROUND OF THE INVENTION

As the number of people routinely living into their eighth decade and beyond rises, the incidence of ageing-related diseases has significantly increased. For example, skeletal muscle atrophy and dysfunction (sarcopenia) has become an increasing age-related health problem, with economic and social consequences (Janssen, I. et al. J. Am. Geriatr. Soc. 52, 80-5 (2004)). This is matched by neuromuscular decline, including an increased prevalence of dementia. To maintain effective performance in any job role attainment of healthy ageing is essential. Furthermore, age is a rough but major parameter in most clinical decision making trees. Identifying the molecular processes governing human ageing and longevity are of great medical importance, but there have been few, human based, discoveries mainly due to the inability to effectively account for influential physiological and environmental factors. There are no diagnostics for healthy ageing in humans.

From epidemiological studies, aerobic fitness (often defined as maximal aerobic capacity) has emerged as one of the most consistent and powerful predictors of long-term health and mortality (Blair et al (1989) Jama 262: 2395-2401; Lee et al (2011) Br J Sports Med 45: 504-510) and the present inventor has established that aerobic fitness is substantially determined by genetic factors (Lortie et al (1982) Hum Biol 54: 801-812; Timmons et al (2010) J Appl Physiol 108: 1487-1496). Accurate determination of aerobic fitness in the laboratory, which is time-consuming, costly and unpleasant for the patient, is used to personalize medicinal decision, e.g. determine the appropriateness of cardiac transplantation or some surgical procedures (Myers et al (2013) Circ Heart Fail 6: 211-218; Voduc (2013) Thorac Surg Clin 23: 233-245).

In fact personalized treatment strategies are, slowly, impacting modern medical practice (Vargas et al (2013) PLoS currents, 5; Wiesweg et al (2013) Eur J Cancer 49: 3076-3082). Novel, easy to administer diagnostics that accurately and sensitively predict future health risk or help guide preventative measures would enable the evaluation of tailored treatment strategies for the individual. Such a method or diagnostic would ideally be applied to healthy middle-aged subjects that have not yet developed clinical disease to provide the greatest opportunity to enhance healthy ageing. Personalized treatment strategies are slowly impacting on modern medical practice (Wiesweg et al (2013)), however none yet offer the possibility to personalize advice to tackle the most frequent causes of morbidity.

In the Uppsala Longitudinal Study of Adult Men (ULSAM) it was found that combining easy to measure risk-factors for cardiovascular disease (e.g. blood pressure) with 4 single protein and biochemical measures in older participants without signs of cardiac disease (healthy′) provided a modest improvement in the C-statistic for diagnostic performance (Zethelius et al (2008) N Engl J Med 358: 2107-2116). A greater circulating cystatin-C concentration at baseline, a parameter that informs about renal function (Inker et al (2012) N Engl J Med 367: 20-29), was related to 10 year mortality in participants with pre-existing disease, but is on its own unable to predict cardiovascular deaths in ‘healthy’ older subjects. Thus, the use of novel single molecule biomarkers, in younger or healthy population samples typically offer very modest improvements in the C-statistic (Wallentin et al (2013) PLoS One 8: e78797; Daniels et al (2011) Circulation 123: 2101-2110) over pre-existing disease markers or the use of chronological age (Rohatgi et al (2014) Clin Chem 58: 172-182). Thus to date we still lack powerful diagnostics of ‘healthy ageing’, tests which do not rely on biomarkers of emerging disease, and which could be applied to disease-free middle-aged subjects.

There are numerous challenges to both the development of, and the technical implementation of, diagnostics for personalized medicine (Goldberger and Buxton (2013) JAMA 309: 2559-2560), including economic considerations. Further, there are multiple competing technological platforms that yield plentiful data, but so far progress in integrating divergent data formats to yield robust and sensitive diagnostics for clinical decision making remains slow (Goldberger and Buxton (2013), supra). Personalized approaches to cancer diagnosis and treatment have been influenced by DNA sequence analysis (Tokuda et al (2009) Breast Cancer 16: 295-300; Patnaik et al (2010) Cancer Res 70: 36-45), and cancer arguably represents where the greatest progress has been made in terms of personalized medicine. Genome-wide association analysis has also identified 281 DNA variants which explained a yet to be verified ˜17% of exceptional longevity in humans (Sebastiani et al (2012) PLoS One 7: e29848). The utility of information on DNA sequence variation to guide treatment of cardiovascular disease or neurodegeneration is just being explored (Sawhney et al (2012) Curr Genomics 13: 446-462), however this approach will be severely limited by the total contribution that DNA variants make to the heterogeneity of these types of diseases.

Global RNA (Passtoors et al (2012) PLoS One 7: e27759; Passtoors et al (2013) Aging Cell 12: 24-31; Gheorghe et al (2014) BMC Genomics 15: 132; Phillips et al (2013) PLoS Genet 9: e1003389; Glass et al (2013) Genome Biol 14: R75) and DNA methylation profiling (Christensen et al (2009) PLoS Genet 5: e1000602; Horvath (2013) Genome Biol 14, R115; Bell et al (2012) PLoS Genet 8: e1002629) have been utilised to search for consistent molecular events correlating with age, where samples come from cross-sectional samples spanning 5-8 decades. Such correlation analyses yield highly significant linear associations, yet by design, such models must be influenced by disease as much as the ageing process per se. For example, Hannum et al built a multi-tissue linear model of DNA methylation age-related changes that correlated with chronological age over seven decades (Hannum et al (2013) Mol Cell 49: 359-367). Furthermore, this molecular profile would not, for example, be useful for distinguishing how successful a person was ageing among a group with the same birth-year (Horvath (2013), supra; Hannum et al (2013), supra) as chronological age and methylation status co-vary tightly. Further, detectable changes in methylation would need to precede the emergence of disease by decades for it to be of practical use.

In Alzheimer's disease (AD), non-invasive blood-based diagnostics (protein or RNA) are being developed to complement clinical and brain-imaging diagnosis of AD and dramatically expand the screening capacity of the health services (Hodges, J. Alzheimers. Dis. 33, 737-53 (2013)). At best, blood RNA diagnostics are 75% accurate at distinguishing AD patients from controls, and work best in later stages of the disease. Further, while very expensive MRI based technology may be 85% accurate, epidemiological analysis indicates there is neither the equipment nor skilled work-force capacity to cope with the numbers of people at risk.

There is therefore an urgent need for an accurate molecular diagnostic of healthy physiological age and/or a molecular model of ageing that diverges sufficiently enough from chronological age.

### SUMMARY OF THE INVENTION

The invention relates to the use of one or more genes as a biomarker for predicting the likelihood of an individual developing an ageing-related disease or to assist with the diagnosis of an ageing-related disease, to a method of predicting the likelihood of an individual developing an ageing-related disease or to assist with the diagnosis of an ageing-related disease, to the use of one or more genes for assessing the ageing effect of a test compound, to a method of assessing the ageing effect of a test compound, to test compounds identified by the invention as having an age-regulating effect and to a kit for assessing the ageing effect of a test compound. Furthermore, use of the biomarker is proposed in a method for identifying drug doses in patients, for rationalization of treatment decisions in a clinical setting or for estimating long-term drug safety. Furthermore, use of the biomarker is proposed as a method for stratifying donor organ status to allow the organ to be matched to the most appropriate recipient for a transplantation procedure. Furthermore, the use of the biomarker is proposed as a method to inform on future sporting performance, industrial performance or to more accurately assess life insurance or health care cost premiums.

According to a first aspect of the invention, there is provided the use of one or more analytes selected from the 670 genes listed in Table 1 as a biomarker for predicting the likelihood of an individual developing an ageing-related disease, or having an age-related clinical adverse event, or to assist with the diagnosis of an ageing-related disease.

TABLE 1

| Gene ID | Gene Name | Gene ID | Gene Name |
|---|---|---|---|
| 217700_at | CNPY4 | 230228_at | SSC5D |
| 234495_at | KLK15 | 201806_s_at | ATXN2L |
| 89476_r_at | NPEPL1 | 215377_at | CTBP2 |
| 244707_at | HCN4 AS | 235491_at | ZBTB10 |
| 244193_at | DNAJC22 | 206889_at | PDIA2 |
| 211180_x_at | RUNX1 | 238313_at | 238313_at |
| 243906_at | 243906_at | 218819_at | INTS6 |
| 214213_x_at | LMNA | 219835_at | PRDM8 |
| 217079_at | 217079_at | 229381_at | C1orf64 |
| 220024_s_at | PRX | 230561_s_at | KANSL1L |
| 240116_at | 240116_at | 231268_at | MYBL1 |
| 229047_at | PLEKHB1 | 221758_at | ARMC6 |
| 241427_x_at | FBXW7 | 238916_at | LINC00938 |
| 230044_at | PCYT2 | 210499_s_at | PQBP1 |
| 216327_s_at | SIGLEC8 | 209966_x_at | ESRRG |
| 219967_at | MRM1 | 244218_at | 244218_at |
| 239125_at | SLC25A5 | 205312_at | SPI1 |
| 234748_x_at | KIF20B | 218827_s_at | CEP192 |
| 206080_at | PLCH2 | 214375_at | PPFIBP1 |
| 230345_at | SEMA7A | 227468_at | CPT1C |
| 238046_x_at | 238046_x_at | 212208_at | MED13L |
| 214209_s_at | ABCB9 | 226428_at | TNPO2 |
| 208232_x_at | NRG1 | 230131_x_at | ARSD |
| 221309_at | RBM17 | 238263_at | EPHA1-AS1 |
| 207883_s_at | TFR2 | 228074_at | ITPRIPL2 |
| 218762_at | ZNF574 | 237646_x_at | PLEKHG5 |
| 239523_at | TUSC5 | 202587_s_at | AK1 |
| 240241_at | 240241_at | 222957_at | NEU4 |
| 227563_at | FAM27E3 | 217040_x_at | SOX15 |
| 240325_x_at | SOX30P1 | 233938_at | C11orf86 |
| 228279_s_at | TNK2 | 213177_at | MAPK8IP3 |
| 205050_s_at | MAPK8IP2 | 227772_at | LATS1 |
| 217410_at | AGRN | 211901_s_at | PDE4A |
| 241563_at | RP11-384L8.1 | 210332_at | LOC100134498 |
| 231242_at | BHLHE41 | 205390_s_at | ANK1 |
| 223153_x_at | TMUB1 | 205629_s_at | CRH |
| 226871_s_at | ATG4D | 34408_at | RTN2 |
| 239837_at | ADAM11 | 206827_s_at | TRPV6 |
| 214316_x_at | CALR | 241921_x_at | 241921_x_at |
| 209983_s_at | NRXN2 | 239251_at | 239251_at |
| 222197_s_at | LOC100128008 | 230046_at | AC005789.11 |
| 233894_x_at | COL26A1 | 238849_at | ACY1 |
| 209097_s_at | JAG1 | 225612_s_at | B3GNT5 |
| 220849_at | EPN2 | 219893_at | CCDC71 |
| 230576_at | BLOC1S3 | 243239_at | SAMM50 |
| 203842_s_at | MAPRE3 | 232568_at | MGC24103 |
| 212512_s_at | CARM1 | 204249_s_at | LMO2 |
| 235879_at | MBNL1 | 216647_at | TCF3 |
| 227287_at | CITED2 | 221493_at | TSPYL1 |
| 207914_x_at | EVX1 | 237144_at | LTBP3 |
| 236845_at | TRIM62 | 218834_s_at | TMEM132A |
| 238406_x_at | SEZ6L2 | 232012_at | CAPN1 |
| 213433_at | ARL3 | 215492_x_at | PTCRA |
| 240686_x_at | TFRC | 34031_i_at | KRIT1 |
| 210364_at | SCN2B | 226675_s_at | MALAT1 |
| 231402_at | LOC100129105 | 226907_at | PPP1R14C |
| 226706_at | FAM20C | 239356_at | LOC100129122 |
| 234342_at | 234342_at | 1569006_at | CTB-167G5.5 |
| 239060_at | 239060_at | 205075_at | SERPINF2 |
| 244182_at | 244182_at | 233073_at | 233073_at |
| 219756_s_at | POF1B | 238866_at | C19orf68 |
| 236269_at | ZNF628 | 215058_at | DENND5B |
| 234400_at | 234400_at | 230625_s_at | TSPAN12 |
| 210483_at | TNFRSF10C | 241211_at | 241211_at |
| 211837_s_at | PTCRA | 239152_at | 239152_at |
| 213987_s_at | CDK13 | 217203_at | GLUL |
| 202588_at | AK1 | 234021_at | EML2 |
| 203876_s_at | MMP11 | 230907_at | GPRC5C |
| 220529_at | FLJ11710 | 212177_at | SFRS18 |
| 204362_at | SKAP2 | 207468_s_at | SFRP5 |
| 236278_at | HIST1H3E | 231480_at | SLC6A19 |
| 231520_at | SLC35F3 | 234746_at | 234746_at |
| 217046_s_at | AGER | 206620_at | CRAP |
| 230375_at | PNISR | 229341_at | TFCP2L1 |
| 240098_at | RIF1 | 234491_s_at | SAV1 |
| 239522_at | IL12RB1 | 215979_s_at | SLC7A1 |
| 225693_s_at | CAMTA1 | 215676_at | BRF1 |
| 239422_at | GPC2 | 237534_at | 237534_at |
| 237046_x_at | IL34 | 53071_s_at | OGFOD3 |
| 228876_at | BAIAP2L2 | 226359_at | GTPBP1 |
| 244591_x_at | RNF207 | 240051_at | TPD52L3 |
| 227211_at | PHF19 | 225571_at | LIFR |
| 221589_s_at | ALDH6A1 | 208661_s_at | TTC3 |
| 204974_at | RAB3A | 213321_at | BCKDHB |
| 234003_at | ENOX2 | 1554274_a_at | SSH1 |
| 214125_s_at | NENF | 207274_at | CHRNE |
| 225072_at | ZCCHC3 | 235432_at | NPHP3 |
| 234536_at | SARDH | 227391_x_at | LRRFIP1 |
| 215026_x_at | SCNN1A | 221136_at | GDF2 |
| 217696_at | FUT7 | 203203_s_at | KRR1 |
| 206906_at | ICAM5 | 225428_s_at | DDX54 |
| 230693_at | ATP2A1 | 213956_at | CEP350 |
| 217074_at | SMOX | 212845_at | SAMD4A |
| 229508_at | U2AF2 | 211119_at | ESR2 |
| 223137_at | ZDHHC4 | 235916_at | YPEL4 |
| 234694_at | CNTROB | 205586_x_at | VGF |
| 220096_at | RNASET2 | 213939_s_at | RUFY3 |
| 208129_x_at | RUNX1 | 242503_at | CHST13 |
| 226141_at | CCDC149 | 202482_x_at | RANBP1 |
| 222080_s_at | SIRT5 | 219636_s_at | ARMC9 |
| 241789_at | RBMS3 | 236479_at | SCN8A |
| 203055_s_at | ARHGEF1 | 244212_at | 244212_at |
| 213690_s_at | 213690_s_at | 231974_at | MLL2 |
| 215488_at | 215488_at | 202401_s_at | SRF |
| 239446_x_at | DCBLD2 | 201882_x_at | B4GALT1 |
| 227781_x_at | FAM57B | 231161_x_at | 231161_x_at |
| 231764_at | CHRAC1 | 222560_at | LANCL2 |
| 219737_s_at | PCDH9 | 221754_s_at | CORO1B |
| 229730_at | SMTNL2 | 237463_at | ZFPM1 |
| 213052_at | PRKAR2A | 209202_s_at | EXTL3 |
| 227720_at | ANKRD13B | 202700_s_at | TMEM63A |
| 204731_at | TGFBR3 | 234411_x_at | CD44 |
| 220482_s_at | SERGEF | 231728_at | CAPS |
| 215649_s_at | MVK | 204104_at | SNAPC2 |
| 238125_at | ADAMTS16 | 223004_s_at | TIMMDC1 |
| 244164_at | FAM223B | 209992_at | PFKFB2 |
| 219150_s_at | ADAP1 | 214312_at | FOXA2 |
| 220989_s_at | AMN | 208607_s_at | SAA1 |
| 205224_at | SURF2 | 213922_at | TTBK2 |
| 206416_at | ZNF205 | 239643_at | RP13-616I3.1 |
| 239629_at | CFLAR | 227520_at | CXorf15 |
| 242197_x_at | CD36 | 203437_at | TMEM11 |
| 1556095_at | UNC13C | 225639_at | SKAP2 |
| 229343_at | GTSE1 | 212771_at | FAM171A1 |
| 216980_s_at | SPN | 214798_at | ATP2C2 |
| 236091_at | HMGB2 | 240624_x_at | LOC100134685 |
| 209280_at | MRC2 | 232534_at | LIN37 |
| 228684_at | ZNF503 | 201452_at | RHEB |
| 229607_at | LOC100652912 | 229714_at | HS6ST3 |
| 218063_s_at | CDC42EP4 | 232480_at | FLJ27365 |
| 212114_at | ATXN7L3B | 221333_at | FOXP3 |
| 240147_at | C7ORF50 | 234714_x_at | ATP2B2 |
| 223426_s_at | EPB41L4B | 209765_at | ADAM19 |
| 202312_s_at | COL1A1 | 229335_at | CADM4 |
| 235671_at | 235671_at | 225290_at | ETNK1 |
| 226674_at | SHISA4 | 205640_at | ALDH3B1 |
| 227456_s_at | C6orf136 | 206646_at | GLI1 |
| 231199_at | RP11-271C24.3 | 226439_s_at | NBEA |
| 244504_x_at | ARF1 | 201300_s_at | PRNP |
| 236030_at | RCOR2 | 203792_x_at | PCGF2 |
| 238006_at | SIN3A | 242744_s_at | CASR |
| 212649_at | DHX29 | 239368_at | 239368_at |
| 228677_s_at | RASAL3 | 214037_s_at | CCDC22 |
| 201592_at | EIF3H | 202305_s_at | FEZ2 |
| 215844_at | TNPO2 | 241894_at | VMO1 |
| 240550_at | OTUB2 | 225545_at | EEF2K |
| 227738_s_at | ARMC5 | 223464_at | OSBPL5 |
| 236746_at | GALNT1 | 237334_at | SFXN2 |
| 224886_at | JMJD8 | 211322_s_at | SARDH |
| 223415_at | RPP25 | 206820_at | AGFG2 |
| 222323_at | CRYGEP | 222346_at | LAMA1 |
| 244566_at | 244566_at | 237764_at | AC062017.1 |
| 241618_at | 241618_at | 1558747_at | SMCHD1 |
| 216289_at | GPR144 | 241125_at | 241125_at |
| 230474_at | UBIAD1 | 206179_s_at | TPPP |
| 208102_s_at | PSD | 239555_at | 239555_at |
| 213170_at | GPX7 | 202005_at | ST14 |
| 224003_at | TTTY14 | 203124_s_at | SLC11A2 |
| 232394_at | RP11-517C16.2 | 1552343_s_at | PDE7A |
| 243567_at | 243567_at | 201921_at | GNG10 |
| 239508_x_at | CCDC108 | 201750_s_at | ECE1 |
| 1556096_s_at | UNC13C | 231030_at | LOC100132618 |
| 241795_at | RHEB | 214917_at | PRKAA1 |
| 228405_at | RHPN1 | 235047_x_at | NACC1 |
| 236885_at | MEX3A | 212417_at | SCAMP1 |
| 232091_s_at | ZDHHC24 | 229112_at | SIRT5 |
| 231224_x_at | PRKAG2 | 238080_at | B4GALNT4 |
| 204375_at | CLSTN3 | 205212_s_at | ACAP1 |
| 211638_at | IGHA1 | 215695_s_at | GYG2 |
| 241961_at | SRD5A2L2 | 210613_s_at | SYNGR1 |
| 225239_at | NEAT1 | 238082_at | 238082_at |
| 1568248_x_at | SNORA71B | 219694_at | FAM105A |
| 234010_at | 234010_at | 217081_at | OR2H2 |
| 207005_s_at | BCL2 | 1556136_at | MYLK4 |
| 230368_at | ERF | 224431_s_at | SUV420H2 |
| 214105_at | SOCS3 | 240210_at | ATAD3C |
| 222543_at | DERL1 | 244057_s_at | VSTM4 |
| 214122_at | PDLIM7 | 240875_at | CTC1 |
| 241629_at | 241629_at | 224932_at | CHCHD10 |
| 237370_at | 237370_at | 227989_at | LTBP4 |
| 206146_s_at | RHAG | 229719_s_at | DERL3 |
| 209266_s_at | SLC39A8 | 213345_at | NFATC4 |
| 234280_at | REG3A | 229353_s_at | NUCKS1 |
| 231561_s_at | APOC2 | 230429_at | 230429_at |
| 222066_at | EPB41L1 | 233128_at | 233128_at |
| 231998_at | SART1 | 237013_at | 237013_at |
| 1558678_s_at | MALAT1 | 242457_at | 242457_at |
| 215661_at | MAST2 | 227991_x_at | ZBTB43 |
| 209971_x_at | JTV1 | 207434_s_at | FXYD2 |
| 243260_x_at | C8orf5 | 207532_at | CRYGD |
| 209446_s_at | PKM2 | 218045_x_at | PTMS |
| 243029_at | KREMEN1 | 223266_at | STRADB |
| 214471_x_at | LHB | 211252_x_at | PTCRA |
| 236348_at | TMEM176B | 213306_at | MPDZ |
| 234918_at | GLTSCR2 | 210783_x_at | CLEC11A |
| 211733_x_at | SCP2 | 204837_at | MTMR9 |
| 235929_s_at | RP11- | 209442_x_at | ANK3 |
| | 399K21.13 | 243285_at | LOC283335 |
| 238325_s_at | ODF3B | 210126_at | PSG9 |
| 218707_at | ZNF444 | 228625_at | CITED4 |
| 211476_at | MYOZ2 | 206278_at | PTAFR |
| 234928_x_at | RUNX3 | 244104_at | MGAT3 |
| 217511_at | KAZALD1 | 217898_at | EMC7 |
| 230170_at | OSM | 208874_x_at | PPP2R4 |
| 221557_s_at | LEF1 | 222040_at | HNRNPA1 |
| 203986_at | STBD1 | 213971_s_at | SUZ12 |
| 216256_at | GRM8 | 202571_s_at | DLGAP4 |
| 223147_s_at | WDR33 | 224996_at | ASPH |
| 228219_s_at | UPB1 | 237075_at | AC104653.1 |
| 213700_s_at | PK | 222667_s_at | ASH1L |
| 239933_x_at | CCDC176 | 228319_at | FAM84A |
| 241671_x_at | CASC15 | 203891_s_at | DAPK3 |
| 208104_s_at | TSC22D4 | 223554_s_at | RANGRF |
| 209979_at | ADARB1 | 200686_s_at | SFRS11 |
| 241670_x_at | LOC729177 | 237454_at | 237454_at |
| 211357_s_at | ALDOB | 212487_at | GPATCH8 |
| 1559641_at | 1559641_at | 240280_at | UFSP1 |
| 236303_at | ARF3 | 208809_s_at | C6orf62 |
| 211576_s_at | SLC19A1 | 230580_at | 230580_at |
| 229434_at | 229434_at | 207643_s_at | TNFRSF1A |
| 202138_x_at | AIMP2 | 224731_at | HMGB1 |
| 236317_at | 236317_at | 227259_at | CD47 |
| 243267_x_at | 243267_x_at | 204144_s_at | PIGQ |
| 229758_at | TIGD5 | 223970_at | RETNLB |
| 227684_at | S1PR2 | 231710_at | CAPS |
| 236744_at | PHPT1 | 229483_at | 229483_at |
| 212958_x_at | PAM | 239689_at | 239689_at |
| 216821_at | KRT8 | 229709_at | ATP1B3 |
| 207025_at | GJC2 | 229638_at | IRX3 |
| 205424_at | TBKBP1 | 215111_s_at | TSC22D1 |
| 206338_at | ELAVL3 | 225807_at | JUB |
| 221013_s_at | APOL2 | 214142_at | ZG16 |
| 206763_at | FKBP6 | 229693_at | TMEM220 |
| 236904_x_at | TECTA | 226400_at | CDC42 |
| 216180_s_at | SYNJ2 | 228651_at | VWA1 |
| 206824_at | CES4 | 244279_at | SOBP |
| 234496_x_at | NYX | 1553702_at | ZNF697 |
| 222154_s_at | SPATS2L | 225874_at | FAM100A |
| 229519_at | FXR1 | 230384_at | ANKRD23 |
| 243651_at | CPEB3 | 227455_at | C6orf136 |
| 221968_s_at | ZNF771 | 206349_at | LGI1 |
| 242287_at | CLIP1 | 231818_x_at | SLC20A2 |
| 226846_at | PHYHD1 | 232323_s_at | TTC17 |
| 230466_s_at | 230466_s_at | 203282_at | GBE1 |
| 231558_at | 231558_at | 210201_x_at | BIN1 |
| 218606_at | ZDHHC7 | 239920_at | UBTF |
| 213389_at | ZNF592 | 202146_at | IFRD1 |
| 218235_s_at | UTP11L | 217858_s_at | ARMCX3 |
| 209359_x_at | RUNX1 | 213976_at | CIZ1 |
| 241929_at | 241929_at | 37831_at | SIPA1L3 |
| 235817_at | TMEM184A | 239613_at | 239613_at |
| 225709_at | ARL6IP6 | 220641_at | NOX5 |
| 213693_s_at | MUC1 | 236318_x_at | FBLL1 |
| 231108_at | FUS | 236689_at | RNF151 |
| 201963_at | ACSL1 | 232933_at | KIAA1656 |
| 201424_s_at | CUL4A | 230247_at | 230247_at |
| 209697_at | 209697_at | 213125_at | OLFML2B |
| 215256_x_at | SNX26 | 230374_at | PPP1R14B |
| 223795_at | TSPAN10 | 226903_s_at | SLC6A10P |
| 222228_s_at | ALKBH4 | 216214_at | 216214_at |
| 234380_x_at | LOC728649 | 207106_s_at | LTK |
| 219417_s_at | C17orf59 | 223956_at | TMPRSS13 |
| 227362_at | SLC2A4RG | 207339_s_at | LTB |
| 213011_s_at | TPI1 | 201140_s_at | RAB5C |
| 228105_at | 228105_at | 208450_at | LGALS2 |
| 217058_at | GNAS | 236356_at | NDUFS1 |
| 213156_at | 213156_at | 214911_s_at | BRD2 |
| 223151_at | DCUN1D5 | 207105_s_at | PIK3R2 |
| 206986_at | FGF18 | 213517_at | PCBP2 |
| 230035_at | BOC | 212331_at | RBL2 |
| 225480_at | C1orf122 | 212205_at | H2AFV |
| 214335_at | RPL18 | 212705_x_at | PNPLA2 |
| 236737_at | ENTHD2 | 230745_s_at | TOX3 |
| 200608_s_at | RAD21 | 233674_at | 233674_at |
| 209449_at | LSM2 | 201374_x_at | PPP2CB |
| 241935_at | SHROOM1 | 230453_s_at | ATP2A3 |
| 208474_at | CLDN6 | 239203_at | LSMEM1 |
| 241799_x_at | 241799_x_at | 221763_at | JMJD1C |
| 242425_at | 242425_at | 235741_at | PPIA |
| 223801_s_at | APOL4 | 224743_at | IMPAD1 |
| 227937_at | MYPOP | 201745_at | TWF1 |
| 208176_at | DUX1 | 232988_at | KIAA0182 |
| 208272_at | RANBP3 | 201557_at | VAMP2 |
| 228823_at | POLR2J2 | 230756_at | ZNF683 |
| 236033_at | ASB12 | 222662_at | PPP1R3B |
| 214056_at | MCL1 | 228231_at | SNX8 |
| 228798_x_at | MAZ | 237018_at | 237018_at |
| 221256_s_at | HDHD3 | 200602_at | APP |
| 216345_at | ZSWIM8 | 239243_at | ZNF638 |
| 229040_at | ITGB2-AS1 | 214024_s_at | DGCR6L |
| 205611_at | TNFSF12 | 219114_at | C3orf18 |
| 235734_at | PACSIN3 | 229198_at | USP35 |
| 231782_s_at | KLK4 | 208615_s_at | PTP4A2 |
| 204692_at | LRCH4 | 214817_at | UNC13A |
| 229717_at | AMIGO3 | 217549_at | 217549_at |
| 242246_x_at | MIR770 | 217231_s_at | MAST1 |
| 211867_s_at | PCDHA10 | 210663_s_at | KYNU |
| 205362_s_at | PFDN4 | 241451_s_at | 241451_s_at |
| 233679_at | MAP3K7IP1 | 232732_at | RP11- |
| 229617_x_at | AP2A1 | | 793H13.3 |
| 239428_at | RAB1A | 217062_at | DMPK |
| 205387_s_at | CGB | 243017_at | USP27X-AS1 |
| 226857_at | ARHGEF19 | 212618_at | ZNF609 |
| 244580_at | 244580_at | 215860_at | SYT12 |
| 201375_s_at | PPP2CB | 211248_s_at | CHRD |
| 215454_x_at | SFTPC | 230531_at | KCNC3 |
| 201996_s_at | SPEN | 219051_x_at | METRN |
| 230439_at | RBAK-RBAKDN | 236439_at | 236439_at |
| 235383_at | MYO7B | 1554171_at | ZMYM3 |
| 236724_at | CFC1 | 234669_x_at | C11orf30 |
| 208412_s_at | RARB | 240949_x_at | 240949_x_at |
| 227294_at | ZNF689 | 201448_at | TIA1 |
| 213740_s_at | TMEM262 | 219654_at | PTPLA |
| 244656_at | RASL10B | 228668_x_at | FLJ36031 |
| 223514_at | CARD11 | 227167_s_at | RASSF3 |
| 207667_s_at | MAP2K3 | 223904_at | PRKAG3 |
| 210393_at | LGR5 | 205332_at | RCE1 |
| 214237_x_at | PAWR | 209262_s_at | NR2F6 |
| 228648_at | LRG1 | 236978_at | 236978_at |
| 230221_at | BAT5 | 225424_at | GPAM |
| 218447_at | CMC2 | 226704_at | UBE2J2 |
| 215367_at | KIAA1614 | 244617_at | GPR26 |
| 203027_s_at | MVD | 229852_at | NMNAT1 |
| 237993_at | CHCHD5 | 237450_at | LOC389332 |
| 236258_at | RBBP8NL | 227662_at | SYNPO2 |
| 241669_x_at | PRKD2 | 210561_s_at | WSB1 |
| 232328_at | ZNF552 | 209850_s_at | CDC42EP2 |
| 239700_at | ZNF710 | 242467_at | 242467_at |
| 215353_at | 215353_at | 219963_at | DUSP13 |
| 205665_at | TSPAN9 | 1553749_at | FAM76B |
| 227935_s_at | PCGF5 | 208470_s_at | HPR |
| 204635_at | RPS6KA5 | 212471_at | AVL9 |
| 205105_at | MAN2A1 | 207353_s_at | HMX1 |
| 238345_at | SLC38A10 | 205714_s_at | ZMYND10 |
| 203996_s_at | C21orf2 | 234795_at | 234795_at |
| 238153_at | PDE6B | 229670_at | 229670_at |

Whilst in principle useful information may be obtained from the levels of expression of individual genes, it has been found that more accurate and reliable information can be obtained by combining information about the levels of expression of each of a panel of several genes, in a linear or non-linear manner.

In one embodiment, all of the 670 genes listed in Table 1 are used as a specific panel of analyte biomarkers for predicting the likelihood of an individual developing an ageing-related disease or to assist with the diagnosis of an ageing-related disease. Information obtained regarding the level of expression of each of the panel of biomarkers may be combined in a linear or non-linear manner.

Data is presented herein which demonstrates a number of advantageous properties for the 670 genes listed in Table 1. For example, the 670 genes were able to distinguish between disease-free old and young brain samples from independent clinical sources and produced under independent laboratory conditions (see Table 7). In addition, the 670 genes demonstrated good classification success in sets of human skin profiles (78%, see Table 7), confirming that the muscle-derived gene-expression signature appears to be a universal diagnostic of human tissue age and able to operate across technology platforms.

The panel of genes may comprise or consist all of the genes identified in Table 1, or at least 30, 50, 70, 100, 120, 130, 140, 150, 200, 300, 500, 600 or 650 of the genes identified in Table 1.

In one embodiment, the panel of genes selected from Table 1 does not include one or more of SKAP2, CEP192, RBM17, NPEPL1, PDLIM7, APP or BIN1. In a further embodiment the panel of genes selected from Table 1 does not include one or more of 1559641_at, 209697_at, 213156_at, 213690_s_at, 215353_at, 215488_at, 216214_at, 217079_at, 217549_at, 228105_at, 229434_at, 229483_at, 229670_at, 230247_at, 230429_at, 230466_s_at, 230580_at, 231161_x_at, 231558_at, 233073_at, 233128_at, 233674_at, 234010_at, 234342_at, 234400_at, 234746_at, 234795_at, 235671_at, 236317_at, 236439_at, 236978_at, 237013_at, 237018_at, 237370_at, 237454_at, 237534_at, 238046_x_at, 238082_at, 238313_at, 239060_at, 239152_at, 239251_at, 239368_at, 239555_at, 239613_at, 239689_at, 240116_at, 240241_at, 240949_x_at, 241125_at, 241211_at, 241451_s_at, 241618_at, 241629_at, 241799_x_at, 241921_x_at, 241929_at, 242425_at, 242457_at, 242467_at, 243267_x_at, 243567_at, 243906_at, 244182_at, 244212_at, 244218_at, 244566_at, or 244580_at.

It has been found that particularly advantageous panels of genes for use in a method of predicting the likelihood of an individual developing an ageing-related disease, or to assist with the diagnosis of an ageing-related disease, comprise at least EIF3H, JMJD8, CDK13, TNK2, TNPO2, CALR, CARM1, NRXN2, RAB3A, SIN3A, TFRC, TGFBR3 and U2AF2. Data is presented herein which demonstrates a number of advantageous properties for such panels of genes. For example, the 13 genes were able to distinguish between old and young muscle tissue and are shown to have utility in distinguishing patients with Alzheimer's Disease (AD) or Mild Cognitive Impairment (MCI) from controls using blood samples. In other embodiments, the panel of genes comprises EIF3H, JMJD8, CDK13, TNK2, TNPO2, CALR, CARM1, NRXN2, RAB3A, SIN3A, TFRC, TGFBR3 and U2AF2 as members of a panel of genes comprising at least 30, at least 50, at least 70, at least 120, or at least 150 of the genes listed in Table 1 or may consist of EIF3H, JMJD8, CDK13, TNK2, TNPO2, CALR, CARM1, NRXN2, RAB3A, SIN3A, TFRC, TGFBR3 and U2AF2 as members of a panel of genes comprising 30, 50, 70, 120, or 150 of the genes listed in Table 1

In a further embodiment, the one or more genes listed in Table 1 are selected from one or more, or each, of ALDH3B1, CAPN1, CDC42EP2, CORO1B, LTBP3, NRXN2, PPP1R14B, RCE1, RCOR2, SART1, SYT12, and ZDHHC24. This embodiment of the invention provides the advantage of representing a panel of genes within the same genomic region, i.e. chromosome 11q13. In another embodiment, the one or more genes listed in Table 1 are selected from one or more, or each, of ALDH3B1, CAPN1, CD44, CDC42EP2, CORO1B, LMO2, LTBP3, NRXN2, PPP1R14B, RCE1, RCOR2, SART1, SYT12, TTC17 and ZDHHC24.

In a further embodiment, the one or more genes listed in Table 1 are selected from one or more, or each, of FXYD2, SCN2B and TMPRSS13. This embodiment of the invention provides the advantage of representing a panel of genes within the same genomic region, i.e. chromosome 11q23.

In one embodiment, the genes are selected from the 150 genes listed in Table 2. Thus, according to a further aspect of the invention, there is provided the use of one or more analytes selected from the 150 genes listed in Table 2 as a biomarker for predicting the likelihood of an individual developing an ageing-related disease or having an age-related clinical adverse event, or to assist with the diagnosis of an ageing-related disease.

TABLE 2

| Gene ID | Gene Name | Gene ID | Gene Name |
|---|---|---|---|
| 217700_at | CNPY4 | 239522_at | IL12RB1 |
| 234495_at | KLK15 | 225693_s_at | CAMTA1 |
| 89476_r_at | NPEPL1 | 239422_at | GPC2 |
| 244707_at | HCN4 AS | 237046_x_at | IL34 |
| 244193_at | DNAJC22 | 228876_at | BAIAP2L2 |
| 211180_x_at | RUNX1 | 244591_x_at | RNF207 |
| 243906_at | 243906_at | 227211_at | PHF19 |
| 214213_x_at | LMNA | 221589_s_at | ALDH6A1 |
| 217079_at | 217079_at | 204974_at | RAB3A |
| 220024_s_at | PRX | 234003_at | ENOX2 |
| 240116_at | 240116_at | 214125_s_at | NENF |
| 229047_at | PLEKHB1 | 225072_at | ZCCHC3 |
| 241427_x_at | FBXW7 | 234536_at | SARDH |
| 230044_at | PCYT2 | 215026_x_at | SCNN1A |
| 216327_s_at | SIGLEC8 | 217696_at | FUT7 |
| 219967_at | MRM1 | 206906_at | ICAM5 |
| 239125_at | SLC25A5 | 230693_at | ATP2A1 |
| 234748_x_at | KIF20B | 217074_at | SMOX |
| 206080_at | PLCH2 | 229508_at | U2AF2 |
| 230345_at | SEMA7A | 223137_at | ZDHHC4 |
| 238046_x_at | 238046_x_at | 234694_at | CNTROB |
| 214209_s_at | ABCB9 | 220096_at | RNASET2 |
| 208232_x_at | NRG1 | 208129_x_at | RUNX1 |
| 221309_at | RBM17 | 226141_at | CCDC149 |
| 207883_s_at | TFR2 | 222080_s_at | SIRT5 |
| 218762_at | ZNF574 | 241789_at | RBMS3 |
| 239523_at | TUSC5 | 203055_s_at | ARHGEF1 |
| 240241_at | 240241_at | 213690_s_at | 213690_s_at |
| 227563_at | FAM27E3 | 215488_at | 215488_at |
| 240325_x_at | SOX30P1 | 239446_x_at | DCBLD2 |
| 228279_s_at | TNK2 | 227781_x_at | FAM57B |
| 205050_s_at | MAPK8IP2 | 231764_at | CHRAC1 |
| 217410_at | AGRN | 219737_s_at | PCDH9 |
| 241563_at | RP11-384L8.1 | 229730_at | SMTNL2 |
| 231242_at | BHLHE41 | 213052_at | PRKAR2A |
| 223153_x_at | TMUB1 | 227720_at | ANKRD13B |
| 226871_s_at | ATG4D | 204731_at | TGFBR3 |
| 239837_at | ADAM11 | 220482_s_at | SERGEF |
| 214316_x_at | CALR | 215649_s_at | MVK |
| 209983_s_at | NRXN2 | 238125_at | ADAMTS16 |
| 222197_s_at | LOC100128008 | 244164_at | FAM223B |
| 233894_x_at | COL26A1 | 219150_s_at | ADAP1 |
| 209097_s_at | JAG1 | 220989_s_at | AMN |
| 220849_at | EPN2 | 205224_at | SURF2 |
| 230576_at | BLOC1S3 | 206416_at | ZNF205 |
| 203842_s_at | MAPRE3 | 239629_at | CFLAR |
| 212512_s_at | CARM1 | 242197_x_at | CD36 |
| 235879_at | MBNL1 | 1556095_at | UNC13C |
| 227287_at | CITED2 | 229343_at | GTSE1 |
| 207914_x_at | EVX1 | 216980_s_at | SPN |
| 236845_at | TRIM62 | 236091_at | HMGB2 |
| 238406_x_at | SEZ6L2 | 209280_at | MRC2 |
| 213433_at | ARL3 | 228684_at | ZNF503 |
| 240686_x_at | TFRC | 229607_at | LOC100652912 |
| 210364_at | SCN2B | 218063_s_at | CDC42EP4 |
| 231402_at | LOC100129105 | 212114_at | ATXN7L3B |
| 226706_at | FAM20C | 240147_at | C7ORF50 |
| 234342_at | 234342_at | 223426_s_at | EPB41L4B |
| 239060_at | 239060_at | 202312_s_at | COL1A1 |
| 244182_at | 244182_at | 235671_at | 235671_at |
| 219756_s_at | POF1B | 226674_at | SHISA4 |
| 236269_at | ZNF628 | 227456_s_at | C6orf136 |
| 234400_at | 234400_at | 231199_at | RP11-271C24.3 |
| 210483_at | TNFRSF10C | 244504_x_at | ARF1 |
| 211837_s_at | PTCRA | 236030_at | RCOR2 |
| 213987_s_at | CDK13 | 238006_at | SIN3A |
| 202588_at | AK1 | 212649_at | DHX29 |
| 203876_s_at | MMP11 | 228677_s_at | RASAL3 |
| 220529_at | FLJ11710 | 201592_at | EIF3H |
| 204362_at | SKAP2 | 215844_at | TNPO2 |
| 236278_at | HIST1H3E | 240550_at | OTUB2 |
| 231520_at | SLC35F3 | 227738_s_at | ARMC5 |
| 217046_s_at | AGER | 236746_at | GALNT1 |
| 230375_at | PNISR | 224886_at | JMJD8 |
| 240098_at | RIF1 | 223415_at | RPP25 |

In one embodiment, all of the 150 genes listed in Table 2 are used as a specific panel of analyte biomarkers for predicting the likelihood of an individual developing an ageing-related disease or to assist with the diagnosis of an ageing-related disease.

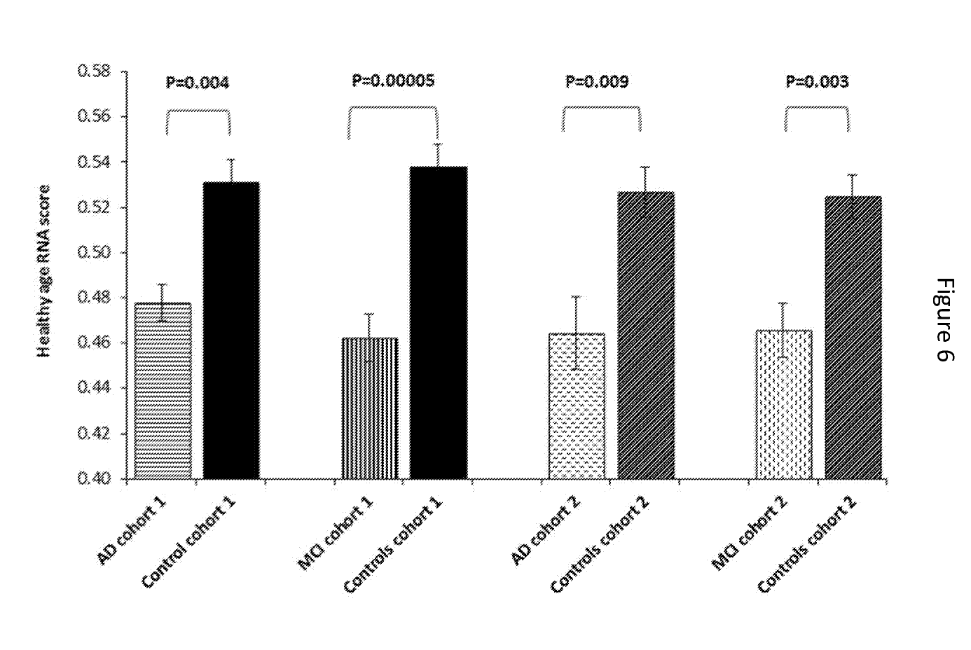

Data is presented herein which demonstrates a number of advantageous properties for the 150 genes listed in Table 2. For example, it was found that use of the 150 genes listed in Table 2 enabled the prediction of 20 year survival (p=0.025) in a cox-regression model, with gene score as a continuous variable. It was also found that healthy controls had a significantly higher gene rank score using the 150 genes listed in Table 2 than subjects with cognitive impairment (FIG. 6).

Preferably, the panel of genes may comprise all of the genes identified in Table 2, or at least 30, 50, 70, 100, 120, 130, 140, 145 or 149 of the genes identified in Table 2, or consist of 30, 50, 70, 100, 120, 130, 140, 145, 149 or 150 of the genes identified in Table 2. In other embodiments, the panel of genes comprises EIF3H, JMJD8, CDK13, TNK2, TNPO2, CALR, CARM1, NRXN2, RAB3A, SIN3A, TFRC, TGFBR3 and U2AF2 as members of a panel of genes comprising at least 30, at least 50, at least 70, or at least 120, of the genes listed in Table 2 or may consist of EIF3H, JMJD8, CDK13, TNK2, TNPO2, CALR, CARM1, NRXN2, RAB3A, SIN3A, TFRC, TGFBR3 and U2AF2 as members of a panel of genes comprising 30, 50, 70, or 120 of the genes listed in Table 2.

In one embodiment, the panel of genes selected from Table 2 does not include one or more of SKAP2, RBM17, or NPEPL1. In a further embodiment the panel of genes selected from Table 2 does not include one or more of 213690_s_at, 215488_at, 217079_at, 234342_at, 234400_at, 235671_at, 238046_x_at, 239060_at, 240116_at, 240241_at, 243906_at or 244182_at.

In one embodiment, the analytes are selected from the 30 genes listed in Table 3. The analytes of this embodiment provide the advantage of yielding an optimised n=30 gene diagnostic for gene-score versus renal function at 82 years (see the data provided herein). Thus, according to a further aspect of the invention, there is provided the use of one or more analytes selected from the 30 genes listed in Table 3 as a biomarker for predicting the likelihood of an individual developing an ageing-related disease or having an age-related clinical adverse event, or to assist with the diagnosis of an ageing-related disease, such as a renal related disease or disorder or a disease characterized by a deterioration in renal function.

TABLE 3

| Gene ID | Gene Name | Affymetrix EXON chip ID |
|---|---|---|
| 223554_s_at | RANGRF | 3709590 |
| 205640_at | ALDH3B1 | 3379305 |
| 229730_at | SMTNL2 | 3742194 |
| 201300_s_at | PRNP | 3874751 |
| 234918_at | GLTSCR2 | 3837464 |
| 241211_at | 241211_at | 3451787 |
| 220024_s_at | PRX | 2722787 |
| 206906_at | ICAM5 | 3850187 |
| 236303_at | ARF3 | 3413680 |
| 232568_at | MGC24103 | 3163530 |
| 231520_at | SLC35F3 | 2461457 |
| 216289_at | GPR144 | 3188780 |
| 202138_x_at | AIMP2 | 2988882 |
| 218045_x_at | PTMS | 3442306 |
| 223147_s_at | WDR33 | 2504766 |
| 232732_at | RP11-793H13.3 | 3898694 |
| 236278_at | HIST1H3E | 2899233 |
| 213987_s_at | CDK13 | 3047189 |
| 220096_at | RNASET2 | 2984884 |
| 224003_at | TTTY14 | 3422257 |
| 208661_s_at | TTC3 | 3931320 |
| 235383_at | MYO7B | 2574966 |
| 215661_at | MAST2 | 2410468 |
| 231782_s_at | KLK4 | 3868728 |
| 203986_at | STBD1 | 2774117 |
| 225072_at | ZCCHC3 | 3894128 |
| 232480_at | FLJ27365 | 3948898 |
| 212417_at | SCAMP1 | 2817053 |
| 215454_x_at | SFTPC | 3089192 |
| 206646_at | GLI1 | 3418120 |

In one embodiment, all of the 30 genes listed in Table 3 are used as a specific panel of analyte biomarkers for predicting the likelihood of an individual developing an ageing-related disease, or to assist with the diagnosis of an ageing-related disease.

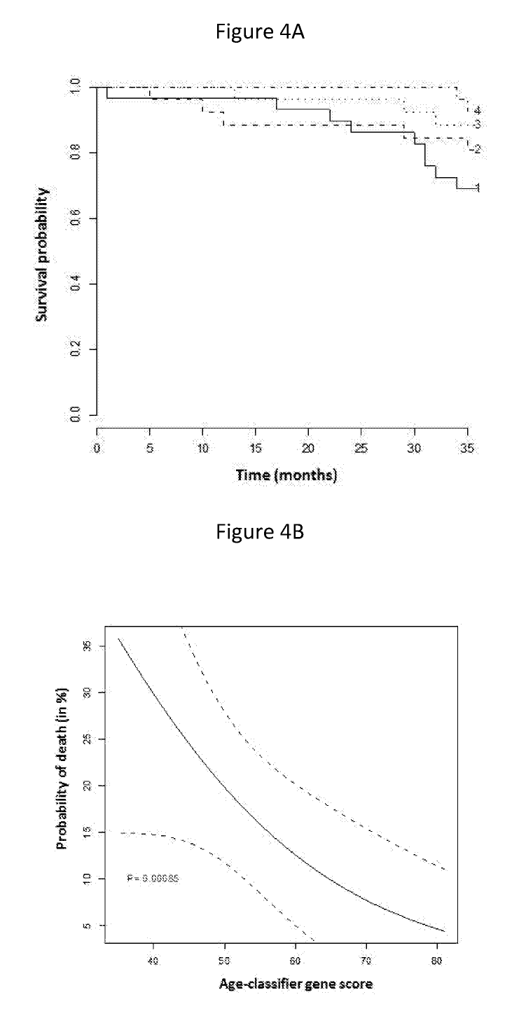

In one embodiment, the analytes are selected from the 30 genes listed in Table 4. The analytes of this embodiment provide the advantage of yielding a strong diagnostic of mortality as demonstrated by logistic regression analysis of gene-score (continuous variable) versus mortality, where a four-fold range in gene-score alone related to up to a 70% probability of death during the 20 year follow-up period (see data presented herein, in particular FIG. 4A). Thus, according to a further aspect of the invention, there is provided the use of one or more analytes selected from the 30 genes listed in Table 4 as a biomarker for predicting the likelihood of an individual developing an ageing-related disease or having an age-related clinical adverse event, such as a disease or disorder likely to result in death of the individual, or to assist with the diagnosis of an ageing-related disease.

TABLE 4

| Gene ID | Gene Name | Affymetrix EXON chip ID |
|---|---|---|
| 209765_at | ADAM19 | 2837413 |
| 201921_at | GNG10 | 3362636 |
| 203055_s_at | ARHGEF1 | 3626426 |
| 230035_at | BOC | 2689034 |
| 220024_s_at | PRX | 2722787 |
| 203027_s_at | MVD | 3673597 |
| 213170_at | GPX7 | 2336439 |
| 212649_at | DHX29 | 2857131 |
| 205586_x_at | VGF | 3400621 |
| 230576_at | BLOC1S3 | 3836135 |
| 226706_at | FAM20C | 3034889 |
| 234928_x_at | RUNX3 | 2325665 |
| 218045_x_at | PTMS | 3442306 |
| 205362_s_at | PFDN4 | 3222991 |
| 204104_at | SNAPC2 | 3819312 |
| 221493_at | TSPYL1 | 2922624 |
| 239920_at | UBTF | 3758967 |
| 212208_at | MED13L | 3433369 |
| 214125_s_at | NENF | 2454715 |
| 230384_at | ANKRD23 | 2565532 |
| 213125_at | OLFML2B | 2364003 |
| 242425_at | 242425_at | 2611238 |
| 227211_at | PHF19 | 3187533 |
| 209983_s_at | NRXN2 | 3334682 |
| 243260_x_at | C8orf5 | 3124227 |
| 230375_at | PNISR | 2918542 |
| 201806_s_at | ATXN2L | 2991090 |
| 237534_at | 237534_at | 3056443 |
| 238866_at | C19orf68 | 2976954 |
| 209262_s_at | NR2F6 | 3824146 |

In one embodiment, all of the 30 genes listed in Table 4 are used as a specific panel of analyte biomarkers for predicting the likelihood of an individual developing an ageing-related disease or to assist with the diagnosis of an ageing-related disease.

In one embodiment, the analytes are selected from the 30 genes listed in Table 5. The analytes of this embodiment provide the advantage of having very high specificity and sensitivity. Thus, according to a further aspect of the invention, there is provided the use of one or more analytes selected from the 30 genes listed in Table 5 as a biomarker for predicting the likelihood of an individual developing an ageing-related disease, or having an age-related clinical adverse event, such as a skin related disease (e.g. failed wound healing) or disorder, or to assist with the diagnosis of an ageing-related disease.

TABLE 5

| Gene Name | Gene ID | Illumina Chip ID |
|---|---|---|
| GPATCH8 | 212487_at | ILMN_1764617 |
| MAPK8IP3 | 213177_at | ILMN_1811574 |
| TPPP | 206179_s_at | ILMN_1718687 |
| IMPAD1 | 224743_at | ILMN_1696311 |
| CTBP2 | 215377_at | ILMN_1691294 |
| SIRT5 | 222080_s_at | ILMN_1738983 |
| RAB3A | 204974_at | ILMN_1755369 |
| OLFML2B | 213125_at | ILMN_1765557 |
| GNG10 | 201921_at | ILMN_1652003 |
| RNF207 | 244591_x_at | ILMN_1802203 |
| PPP2R4 | 208874_x_at | ILMN_1652249 |
| U2AF2 | 229508_at | ILMN_1768930 |
| TTC17 | 232323_s_at | ILMN_1660810 |
| NPEPL1 | 89476_r_at | ILMN_1724194 |
| ASPH | 224996_at | ILMN_1693771 |
| PTMS | 218045_x_at | ILMN_1721046 |
| NOX5 | 220641_at | ILMN_1775298 |
| PLEKHG5 | 237646_x_at | ILMN_1765109 |
| AK1 | 202588_at | ILMN_1691736 |
| METRN | 219051_x_at | ILMN_1712583 |
| PRKAG3 | 223904_at | ILMN_1716754 |
| LIFR | 225571_at | ILMN_1709094 |
| MYO7B | 235383_at | ILMN_1793529 |
| B4GALT1 | 201882_x_at | ILMN_1766221 |
| MAP2K3 | 207667_s_at | ILMN_1680777 |
| ABCB9 | 214209_s_at | ILMN_1788928 |
| SSH1 | 1554274_a_at | ILMN_1727671 |
| NRXN2 | 209983_s_at | ILMN_1738684 |
| SKAP2 | 225639_at | ILMN_2125010 |
| MVD | 203027_s_at | ILMN_1657550 |

In one embodiment, all of the 30 genes listed in Table 5 are used as a specific panel of analyte biomarkers for predicting the likelihood of an individual developing an ageing-related disease or to assist with the diagnosis of an ageing-related disease.

Preferably, the panel of genes may comprise all of the genes identified in any one of Table 3, Table 4 or Table 5, or at least 15, 20, 25, or 27 of the genes identified in any one of Table 3, Table 4 or Table 5, or may consist of 15, 20, 25, or 27 of the genes identified in any one of Table 3, Table 4 or Table 5.

References herein to “biomarker” refer to a distinctive biological or biologically derived indicator of a process, event, or condition.

A major advantage of the invention is that the identified biomarkers are not affected by various extraneous physiological factors affecting the biological sample in which the level of analyte biomarkers are measured (such as body mass index, aerobic capacity, impaired glucose tolerance and physical fitness). This has the effect that the ageing signature can be used to accurately predict the likelihood of an individual developing an ageing-related disease in a wider range of test subjects.

It will be appreciated that references herein to “likelihood” refer to the probability that a particular event will occur. The biomarkers of the invention provide a novel way to assess whether an individual has a higher or lower probability, or risk, of developing an ageing-related disease, depending on the expression levels of the biomarkers defined herein.

References herein to “ageing-related disease” refer to various diseases that have been associated with the increasing biological age of an individual. Such diseases can also be referred to as “ageing-associated diseases”, “degenerative diseases” or “diseases of the elderly”. An individual has an increased risk of developing an ageing-related disease as their biological age increases.

Ageing-related diseases include a range of diseases such as, cardiovascular disease, atherosclerosis, coronary heart disease, cardiomyopathy, congestive heart failure, hypertensive heart disease, hypertension, arthritis, osteoarthritis, rheumatoid arthritis, type 2 diabetes, multiple system atrophy, inflammatory bowel disease, Crohn's disease, age-related cancer, shingles, cataracts, glaucoma, age-related macular degeneration, osteoporosis, sarcopenia, fibromyalgia, Parkinson's disease, Alzheimer's disease, dementia, vascular dementia, frontotemporal dementia, progressive dementia, Lewy Body dementia, semantic dementia, mild-cognitive impairment (MCI) and diseases characterised by a deterioration in renal function. Age-related conditions would also include impaired recovery from a surgical intervention, accelerated loss of muscle tissue following a fracture or accident or illness induced bed-rest, susceptibility to impaired wound healing and hence infection, susceptibility for motor-skill impairments and falls.

Further, the severity of conditions that present as a type of accelerated ageing, such as multiple sclerosis, ALS (amyotrophic lateral sclerosis, often referred to as Lou Gehrig's Disease) and laminin related diseases would benefit from a more accurate prognosis of the time-course of the disease, using the diagnostic.

As the incidence of ageing-related diseases increases, along with the increasing strain on the healthcare system, it is advantageous to be able to predict an individual's likelihood of developing an ageing-related disease as this permits initiation of appropriate therapy, or preventive measures, e.g. managing risk factors. This information may also be advantageously be used to select patients to participate in clinical trials who have a higher risk of developing an ageing-related disease.

According to a further aspect of the invention there is provided the use of one or more genes listed in Table 1 or Table 2 or Table 3 or Table 4 or Table 5, or of a panel of genes as defined herein, as a biomarker for assessing the potential duration of a sporting career e.g. Major League Baseball, Grid-Iron or Soccer.

According to a further aspect of the invention there is provided the sum or alternative arithmetic conversion of the levels of expression of 2 or more genes listed in Table 1 or Table 2 or Table 3 or Table 4 or Table 5, or of the level of expression of each of a panel of genes as defined herein, to create a biological (as opposed to a chronological) ageing index for use individually or as a component of a clinical decision making nomogram or decision tree.

According to a further aspect of the invention there is provided the sum or alternative arithmetic conversion of the levels of expression of 2 or more genes listed in Table 1 or Table 2 or Table 3 or Table 4 or Table 5, or of the level of expression of each of a panel of genes as defined herein, to create a biological (as opposed to a chronological) ageing index for use individually or as a component of a decision making nomogram for trading or purchasing professional athletes.

According to a further aspect of the invention there is provided the sum or alternative arithmetic conversion of the levels of expression of 2 or more genes listed in Table 1 or Table 2 or Table 3 or Table 4 or Table 5, or of the level of expression of each of a panel of genes as defined herein, to create a biological (as opposed to a chronological) ageing index for use individually or as a component of a decision making nomogram for estimating insurance costs related to health and life-span.

It has been found that the 670 genes listed in Table 1 were over represented at certain genomic loci. Thus, according to a further aspect of the invention there is provided a method of predicting the likelihood of an individual developing an ageing-related disease or having an age-related clinical adverse event which comprises the step of detecting the presence of a genetic variation or a significant difference in gene expression compared with a control subject within one or more of the following regions of the human genome: 7q22, 11q13 and 11q23. In one embodiment, the region of the human genome is selected from 11q13 and 11q23.

In a further embodiment, the region of the human genome is selected from 11q13 and the method comprises the detection of a genetic variation within one or more, or each, of the following genes: ALDH3B1, CAPN1, CDC42EP2, CORO1B, LTBP3, NRXN2, PPP1R14B, RCE1, RCOR2, SART1, SYT12 and ZDHHC24.

In a further embodiment, the region of the human genome is selected from 11q23 and the method comprises the detection of a genetic variation within one or more, or each, of the following genes: FXYD2, SCN2B and TMPRSS13.

References herein to “genetic variation” include any variation in the native, non-mutant or wild type genetic code of the gene under analysis. Examples of such genetic variations include: mutations (e.g. point mutations), substitutions, deletions, insertions, single nucleotide polymorphisms (SNPs), haplotypes, chromosome abnormalities, Copy Number Variation (CNV), epigenetics and DNA inversions.

According to a further aspect of the invention, there is provided a method of predicting the likelihood of an individual developing an ageing-related disease, or to assist with the diagnosis of an ageing-related disease, or predicting the likelihood of an organ from an individual over >50 years of age being successfully used for transplantation into a donor patient, which comprises the steps of:
(a) quantifying, in a biological sample from the individual, the level of expression of one or more analyte biomarkers as defined herein; and
(b) comparing the level of expression quantified in step (a), with a control level of expression of the one or more analyte biomarkers;
   such that a change in expression is indicative of the individual's risk to developing an ageing-related disease or death, or the presence of the ageing related disease, or of a successful organ transplantation.

Preferably, the level of expression of each of a panel of genes, as defined herein, is quantified in the biological sample from the individual and compared with the control levels of expression for each of the panel of genes. In one embodiment, the panel of genes comprises at least EIF3H, JMJD8, CDK13, TNK2, TNPO2, CALR, CARM1, NRXN2, RAB3A, SIN3A, TFRC, TGFBR3 and U2AF2. In another embodiment, the panel of genes comprises at least EIF3H, JMJD8, CDK13, TNK2, TNPO2, CALR, CARM1, NRXN2, RAB3A, SIN3A, TFRC, TGFBR3 and U2AF2 as members of a panel of genes comprising at least 30, at least 70, at least 120, or at least 150 of the genes listed in Table 1, or at least 30, at least 70, or at least 120 of the genes listed in Table 2. In further embodiments, the panel of genes comprises at least 30 of the 670 genes listed in Table 1, such as at least the 30 genes listed in any one of Table 3, Table 4 and Table 5, or at least 150 of the 670 genes listed in Table 1, such as at least the 150 genes listed in Table 2.

Information from the method of predicting the likelihood of an individual developing an ageing-related disease as defined herein may be used in a method of selecting individuals to participate in a clinical trial, such as a clinical trial to assess the efficacy of a new method of treatment of the ageing-related disease, for example Alzheimer's disease. The information obtained relating to the likelihood of the development of the ageing-related disease for each individual may be used to stratify the individuals, enabling individuals with a high risk of the disease to be selected to participate in the clinical trial. For example, to screen new Alzheimer's disease drugs in 2015, 1 million older people are required to undergo an initial assessment to find the most suitable 100,000. The present method could reduce the initial numbers 500% and so speed up drug development 5-fold.

According to a further aspect of the invention there is provided a method of predicting the likelihood of an individual developing an ageing-related disease, or to assist with the diagnosis of an ageing-related disease, or predicting the likelihood of an organ from an individual over >50 years of age being successfully used for transplantation into a donor patient, which comprises the steps of (i) quantifying, in a biological sample from the individual, the level of expression of each of a panel of genes; and (ii) comparing the levels of expression quantified in step (i), with control levels of expression for each of the panel of genes; such that changes in the levels of expression are indicative of the individual's risk to developing the ageing-related disease or of a successful organ transplantation; and wherein the panel of genes is selected using a method comprising the steps of: (a) obtaining a biological sample from one or more young human subjects; (b) obtaining a biological sample from one or more older human subjects wherein said older human subjects are disease free; (c) conducting gene expression analysis upon each of the samples obtained in steps (a) and (b) and selecting a panel of genes which show a significant difference in gene expression between the samples obtained in steps (a) and (b).

It will be appreciated that the term “quantifying” refers to calculating the amount of analyte biomarker, such as the amount of each of a panel of genes, in a sample. This may include determining the concentration of the analyte biomarker present in a sample. Quantification may be performed directly on the sample, indirectly on an extract therefrom, or on a dilution. In one embodiment, the level of gene expression may be quantified using a method comprising the following steps: (i) reverse transcription of RNA to cDNA; (ii) hybridization with at least one oligonucleotide probe; (iii) quantification of gene expression levels. The method may additionally include the step of labeling the cDNA, for example, prior to hybridization. As an alternative, the oligonucleotide probes may be labelled. The quantification of gene expression levels may be carried out, for example, using an analysis of fluorescence or radioisotope levels, depending on the method of labelling utilized. Quantification may be carried out using at least one DNA microarray, with analysis carried out, for example, utilising a DNA microarray scanner.

Therefore, in a further aspect of the invention there is provided a method of predicting the likelihood of an individual developing an ageing-related disease, or to assist with the diagnosis of an ageing-related disease, or predicting the likelihood of an organ from a person over >50 years of age being successfully used for transplantation into a donor patient, which comprises the steps of:
(a) contacting, under conditions allowing hybridization between complementary sequences, the nucleic acids from a biological sample from a test subject and a panel of probes, the panel of probes, for example, comprising at least 30 of the probe sets identified in Table 1, Table 2, Table 3, Table 4 or Table 5, in order to obtain an expression profile; and
(b) comparing the expression profile generated in step (a), with a control level of expression;
   such that a change in expression is indicative of an individual's risk to developing an ageing-related disease, or the presence of the ageing related disease, or of a successful organ transplantation.

The panel of probes may comprise at least 30, 50, 70, 100, 120, 130, 140, 150, 200, 300, 500, 600 or 650 of the probesets identified in Table 1 (by Gene IDs), or at least 30, 50, 70, 100, 120, 130, 140, 145 or 149 of the probesets identified in Table 2, or at least 15, 20, 25, or 27 of the probesets identified in any one of Table 3, Table 4 or Table 5, or may alternatively comprise probesets with a complementary sequence to the panels of probes defined herein. Preferably, the panel of probes comprises at least the probesets 204974_at, 201592_at, 209983_s_at, 240686_x_at, 238006_at, 229508_at, 214316_x_at, 204731_at, 224886_at, 213987_s_at, 215844_at, 212512_s_at and, 228279_s_at.

The “control level” used in the methods of the invention may be provided as a reference value for the expression level of the chosen analyte, or of each of a panel of analytes, in a test subject of the corresponding age range. A reference value may be devised from a statistical assessment of the expression levels of a particular analyte, or of a panel of analytes, generated from biological samples taken from a plurality or statistically-significant number of test subjects of the corresponding age range. The control level of a particular analyte, or of each of a panel of analytes, may also be derived from externally available gene expression data sets.

In one embodiment, the control level value of a particular analyte, such as each of a panel of analytes, may be generated by measuring the expression level of an analyte defined herein, in skeletal muscle biopsies. In a further embodiment the control level values may be generated from samples obtained from at least 10, at least 20, or in particular at least 30 test subjects of a selected age range.

Human skeletal muscle provides the ideal starting tissue from which to generate a ‘clean’ ageing molecular classifier, as skeletal muscle RNA is easily accessible and its functional status can be studied in great detail prior to tissue sampling in all age groups. This lies in very distinct contrast to using brain, myocardium or any one of a number of other potential human tissue sources because the function of the latter examples can not be measured at the time of tissue sampling.

A change in expression level of the analyte biomarkers defined herein, is indicative of an individual's risk of developing an ageing-related disease. If the ageing signature is opposed or inhibited, i.e. the expression of an analyte which is up-regulated with age is decreased compared to the control value or an analyte which is down-regulated with age is increased compared to the control value, this is indicative of an individual having a greater risk of developing an ageing-related disease, or the presence of the ageing-related disease, or having a higher mortality (FIG. 4B). If the ageing signature is activated or induced, i.e. the expression of an analyte which is up-regulated with age is increased compared to the control value or an analyte which is down-regulated with age is decreased compared to the control value, this is indicative of an individual having activated the ‘healthy age’ programme with the concomitant improved mortality or functional capacity.

The change in expression levels may be assessed, for example, using a gene-ranking approach. Each of the gene expression levels, obtained by quantification of the biological sample from the individual, may be compared with the level of expression of the same gene in each of multiple biological samples taken from multiple different test subjects. The gene expression level may then be ranked in comparison with the levels of expression observed in the samples from test subjects. The order of the ranking takes into account whether the gene is up-regulated or down-regulated during healthy-ageing, such as whether the gene was up-regulated or down-regulated between the young and old samples in the ‘Stockholm’ data set. The rankings of all of the genes of the panel may then be combined, for example using the sum, median, mean or alternative arithmetic conversion.

It is advantageous to be able to assess an individual's biological age accurately, so that if individuals are identified as having a high risk of developing an ageing-related disease they can act accordingly to reduce their risk, such as through lifestyle changes or prophylactic treatment. The analyte biomarkers defined herein have a further advantage because they can provide insight into which physiological traits have potential links to longevity.

In one embodiment the biological sample from the individual and/or the biological sample from the young and/or older human subjects is a tissue sample. This may be a tissue homogenate, tissue section and biopsy specimens taken from a live subject, or taken post-mortem. The samples can be prepared, for example where appropriate diluted or concentrated, and stored in the usual manner.

The analyte biomarkers provided by the invention, have the considerable advantage of accurately predicting the biological ageing in a variety of tissues, and hence the likelihood of an individual developing an ageing-related disease. This allows the method to be carried out on any tissue that is the most cost-effective and readily available.

In a further embodiment the tissue sample is obtained from the skin, hair, oral mucosa, brain, heart, liver, lungs, stomach, pancreas, kidney, bladder, skeletal muscle, cardiac muscle or smooth muscle. In a further embodiment, the tissue sample is obtained from skeletal muscle. In one embodiment, the biological sample is a sample of cells.

In one embodiment the biological sample from the individual and/or the biological sample from the young and/or older human subjects is a blood sample, such as whole blood, blood serum or blood plasma. In one embodiment the quantification of analyte biomarkers is performed using a biosensor.

In one embodiment the ageing-related disease is Alzheimer's disease (AD), mild cognitive impairment (MCI) or dementia. In another embodiment, ageing-related disease is AD, MCI, or dementia and the biological sample from the individual is a blood sample, such as whole blood, blood serum or blood plasma. In a further embodiment, the ageing-related disease is AD, MCI, or dementia, the biological sample from the individual is a blood sample, such as whole blood, blood serum or blood plasma, and the biological sample from the young and older human subjects is a tissue sample obtained from skeletal muscle or skin. It will be appreciated that the use of the analyte biomarkers described herein advantageously provides a diagnostic of cognitive impairment utilizing only peripheral samples. The analyte biomarkers may additionally be combined with alternative diagnostic tests utilising other biomarkers of cognitive impairment, or with diagnostics based on clinical parameters, to enhance the performance of such diagnostics.

It will be appreciated that the methodology of identifying the analyte biomarkers of the invention constitutes a novel and inventive aspect of the invention not used in previous studies. For example, it is common practice to identify an age related biomarker by comparing analyte levels (via gene expression levels) in a sample obtained from a young subject with analyte levels in a sample obtained from an elderly subject. By contrast, the present invention obtained samples from young subjects (i.e. subjects under 28 years of age) and older subjects (i.e. subjects over 59 years of age) who were free from clinical metabolic and cardiovascular disease. In addition, the young and older subjects may be selected to have equivalent aerobic fitness levels as determined using gas analysis and a maximal exercise protocol.

The advantage of the method of the invention is that the genes identified should associate with, or reflect, healthy physiological age rather than disease as older subjects were specifically selected to be disease free.

In one embodiment, the young human subjects are under 30 years of age. In a further embodiment, the young human subjects are between 18 and 30 years of age. In a yet further embodiment, the young human subjects are selected from any one of the following ages: 30, 29, 28, 27, 26, 25, 24, 23, 22, 21, 20, 19 or 18 years of age, such as younger than 28 years of age.

References herein to “disease free” refer to a subject not presenting with any symptoms of a diagnosable disease or disorder. In one embodiment, disease free comprises free from metabolic and cardiovascular disease. In a further embodiment, said older human subjects comprise subjects having a good aerobic fitness and glucose tolerance. Preferably, the young and old subjects are selected to have equivalent aerobic fitness levels as determined using gas analysis and a maximal exercise protocol. In one embodiment, the ageing-related disease is AD or MCI and the older human subjects are free from AD and/or MCI.

In one embodiment, the older human subjects are older than the young human subjects sampled in step (a) of the described aspects of the invention. In a further embodiment, the older human subjects are between 55 and 70 years of age. In a yet further embodiment, the older human subjects are selected from any one of the following ages: 55, 56, 57, 58, 59, 60, 61, 62, 63, 64, 65, 66, 67, 68, 69 or 70 years of age, such as greater than 59 years of age. In another embodiment the young human subjects are under 30 years of age and the older subjects are greater than 59 years of age or the older subjects were between 55 and 70 years of age. In yet another embodiment the young human subjects are between 18 and 30 years of age and the older subjects are between 55 and 70 years of age.

According to a further aspect of the invention there is provided a method of identifying a biomarker for predicting the likelihood of an individual developing an ageing-related disease, or having an age-related clinical adverse event, or to assist with the diagnosis of an ageing-related disease wherein said method comprises the steps of:

(a) obtaining a biological sample from one or more young human subjects;

(b) obtaining a biological sample from one or more older human subjects wherein said older human subjects are disease free;

(c) conducting gene expression analysis upon each of the samples obtained in steps (a) and (b);

wherein a significant difference in gene expression between the samples obtained in steps (a) and (b) is indicative of a biomarker for predicting the likelihood of an individual developing an ageing-related disease, or having an age-related clinical adverse event, or the presence of the ageing related disease.

According to a further aspect of the invention, there is provided a biomarker for predicting the likelihood of an individual developing an ageing-related disease, or having an age-related clinical adverse event, or the presence of the ageing related disease identified by the method defined herein.

In one embodiment, the biomarker is one or more analytes selected from the genes listed in Table 1 or Table 2 or Table 3 or Table 4 or Table 5. Preferably the biomarker is a panel of genes as defined herein.

According to a further aspect of the invention, there is provided a biomarker as defined herein for use in predicting the likelihood of an organ from a person over >50 years of age being successfully used for transplantation into a donor patient. Furthermore, there is provided a biomarker as defined herein for use in a method of stratifying donor organ status to enable matching the organ to the most appropriate recipient for transplantation. In one embodiment, the biomarker is one or more analytes selected from the genes listed in Table 1 or Table 2 or Table 3 or Table 4 or Table 5. Preferably the biomarker is a panel of genes as defined herein.

References herein to “biosensor” refer to anything capable of detecting the presence of the biomarker. For example, the biosensor may comprise a high throughput screening technology, e.g. configured in an array format, such as a chip or as a multi-well array. High-throughput screening technologies are particularly suitable to monitor biomarker signatures for the identification of potentially useful ageing compounds.

A biosensor may also comprise a ligand or ligands capable of specific binding to the analyte biomarker, such as an antibody or biomarker-binding fragment thereof, or other oligonucleotide, or ligand, e.g. aptamer, or peptide, capable of specifically binding the biomarker. The ligand may possess a detectable label, such as a luminescent, fluorescent or radioactive label, and/or an affinity tag.

Suitably, biosensors for detection of one or more biomarkers of the invention combine biomolecular recognition with appropriate means to convert detection of the presence, or quantification, of the biomarker in the sample into a signal. According to a further aspect of the invention, there is provided the use of one or more analytes selected from the genes listed in Table 1 or Table 2 or Table 3 or Table 4 or Table 5, or of a panel of genes as defined herein, as a biomarker for assessing the ageing effect of a test compound.

Analyte biomarkers can be used in, for example, clinical screening, drug screening and development. Biomarkers and uses thereof are important in the identification of novel compounds in in vitro and/or in vivo assays.

The biomarkers described herein may also be referred to collectively as an “ageing molecular classifier”, “healthy ageing diagnostic” or “longevity diagnostic”. They are part of the first accurate multi-tissue molecular classifier of ageing, as supported by the data provided herein.

Therefore, the biomarkers provided by the invention can act as a valuable indicator to establish whether a test compound has an effect on ageing in a variety of tissues. They represent a new resource for developing small-molecule drugs targeted at modifying ageing biology.

The biomarkers described herein can also be used as suitable toxicology biomarkers to be used in drug-safety screening. In particular, they can be used to predict whether a compound will have any long-term side-effects on the premature ageing of a tissue. According to a further aspect of the invention there is therefore provided the use of one or more genes listed in Table 1 or Table 2 or Table 3 or Table 4 or Table 5, or of a panel of genes as defined herein, as a biomarker for assessing the safety effect of a test compound.

Ageing can have an effect upon the physiological condition of a cell, tissue or organism. References herein to “ageing effect” refer to both a pro- and anti-ageing effect. An “anti healthy ageing” effect results when the ageing signature, as described herein, is opposed, whereas a “pro healthy ageing” effect results when the ageing signature is induced. The invention has the advantage of distinguishing whether a test compound has an anti-health, a pro-health or no effect on healthy ageing at all (for drug safety).

References herein to “test compound” can refer to a chemical or pharmaceutical substance to be tested using the analyte biomarkers described herein. The test compound may be a known substance or a novel synthetic or natural chemical entity, or a combination of two or more of the aforesaid substances.

In one embodiment each of the genes listed in Table 1 or Table 2 or Table 3 or Table 4 or Table 5, or a panel of genes as defined herein, are used as a specific panel of analyte biomarkers for assessing the ageing effect of a test compound.

According to a further aspect of the invention, there is provided a method of assessing the ageing effect of a test compound which comprises the steps of:

(a) incubating the test compound with a biological sample;
(b) quantifying the level of expression of one or more of the analyte biomarkers as defined herein; and
(c) comparing the level of expression quantified in step (b), with the level of expression of the one or more analyte biomarkers in said biological sample in the absence of the test compound;
such that a change in expression is indicative of the ageing effect of the test compound.

It will be understood that activation of the health ageing expression pattern is indicative of a test compound having a beneficial effect, whereas inhibition of the health ageing expression pattern is indicative of a test compound having a pro-ageing or unhealthy effect.

The invention described herein, has the advantage of distinguishing whether a compound has a pro healthy ageing or an anti healthy ageing effect in a single procedure, depending on whether the ageing signature is opposed or induced directly in human material. This helps to cut down costs when screening multiple test compounds using accurate, but expensive, microarray technologies.

A further advantage of the invention is that the identified biomarkers are not affected by various extraneous physiological factors affecting the biological sample that the compounds are tested on (such as body mass index, aerobic capacity, impaired glucose tolerance and physical fitness). This indicates that the compounds identified by the analyte biomarkers to have an ageing effect, are more likely to work on a wider range of consumers.

Preferably, the analyte biomarkers are a panel of genes as defined herein.

In one embodiment the biological sample is a tissue sample. This may be a tissue homogenate, tissue section and biopsy specimens taken from a live subject, or taken postmortem. The samples can be prepared, for example where appropriate diluted or concentrated, and stored in the usual manner.

The analyte biomarkers provided by the invention, have the considerable advantage of accurately predicting the ageing effect of a test compound in a variety of tissues. This allows the method to be carried out on any tissue that is the most cost-effective and readily available.

In a further embodiment the tissue sample is obtained from the skin, hair, oral mucosa, brain, heart, liver, lungs, stomach, pancreas, kidney, bladder, skeletal muscle, cardiac muscle or smooth muscle. In a further embodiment, the tissue sample is obtained from skeletal muscle.

In one embodiment, the biological sample is a sample of cells. In a further embodiment the sample of cells is derived from a cancer cell line. Cancer cell lines can be grown reproducibly and stably in a test tube and therefore provides a suitable biological sample to measure the in vitro effect of a test compound on the healthy ageing signature.

In one example, the ageing signature may be measured in a sample of cancer cells obtained from a patient to provide information on the potential aggression of a tumour, or its ability to survive therapy. If the healthy ageing signature is reduced by a chosen therapeutic, then this is indicative of a pro-survival effect on the cancer cells within the target tumour.

In one embodiment the quantification of analyte biomarkers is performed using a biosensor.

A further aspect of the invention provides a method of treating an ageing-related disease in an individual, which comprises assessing the risk of said individual developing an ageing-related disease according to any of the methods defined herein and if the individual is identified as being at risk of developing an ageing-related disease, treating said individual to prevent or reduce the onset of an ageing-related disease.

A further aspect of the invention provides a compound obtainable by the method as defined herein.

Compounds that activate the ageing signature can be considered “pro healthy ageing” compounds and can be used as effective therapeutics. In particular, pro-ageing compounds can provide a novel anti-cancer therapeutic by enhancing surveillance for cancerous tumor cells. In another example, a pro-ageing compound may be used to activate the healthy ageing signature in skin cells to help accelerate wound healing.

Compounds that inhibit the ageing signature can be considered “anti healthy ageing” compounds. Drugs which create this pattern of expression would be important to identify during the drug discovery and development process. In one example an identified anti healthy ageing compound may in the long term damage tissues, such as heart or muscle tissue, and the proposed screen would identify these unwanted and/or negative effect.

In one embodiment, the compound is a nutraceutical compound. References herein to “nutraceutical” refer to any substance that is a food or a part of a food that provides medical or health benefits, including the prevention and treatment of disease. Such products may range from isolated nutrients, dietary supplements and specific diets, to genetically engineered designer foods, herbal products, and processed foods such as cereal, soups and beverages.

According to a further aspect of the invention, there is provided a kit for assessing the ageing effect of a test compound comprising a biosensor capable of quantifying the analyte biomarkers as defined herein. In one embodiment, the kit comprises reagents from the Affymetrix Gene-Chip technology platform.

Suitably a kit according to the invention may contain one or more components selected from the group: a ligand specific for the analyte biomarker or a structural/shape mimic of the analyte biomarker, one or more controls, one or more reagents and one or more consumables. Optionally the kit may be provided with instructions for use of the kit in accordance with any of the methods defined herein.

The present invention will now be illustrated by the following studies, and with reference to the accompanying figures, in which:

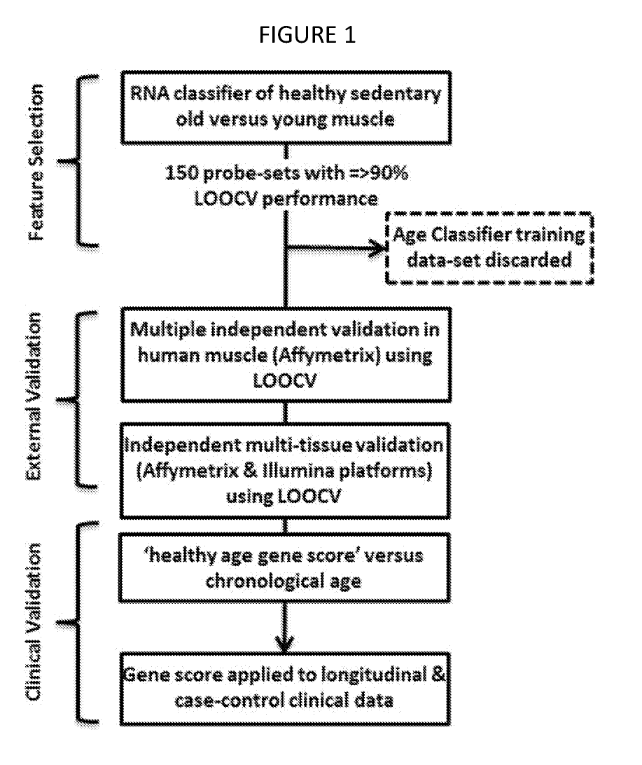

FIG. 1 shows a schematic overview of the use of RNA probe-sets for the development, validation and optimization of the healthy physiological age diagnostics.

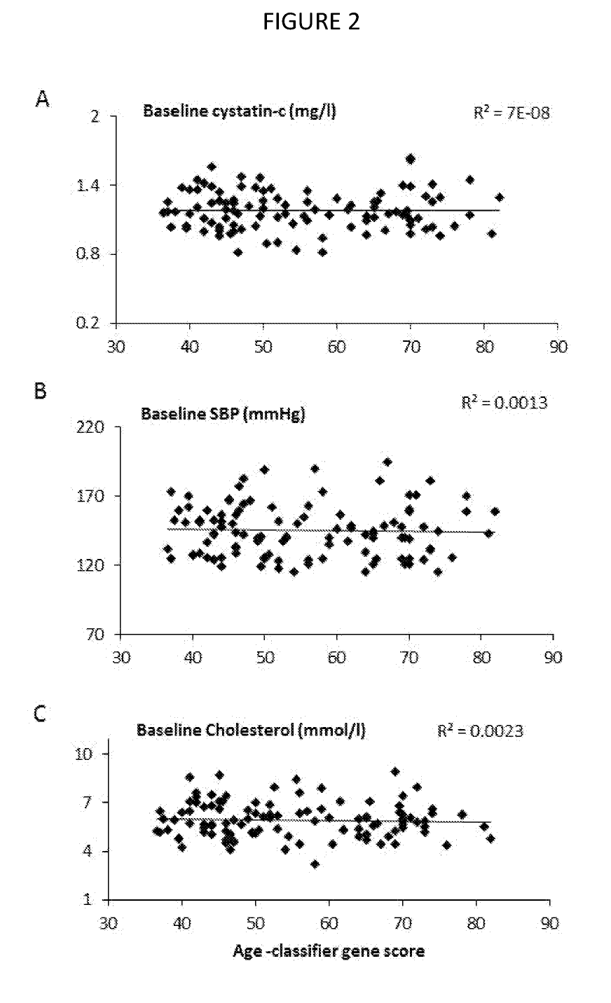

FIG. 2 provides plots of a cumulative gene score calculated using the 150 genes of Table 2 in ULSAM samples (chronological age=69-70y) against conventional clinical risk factors

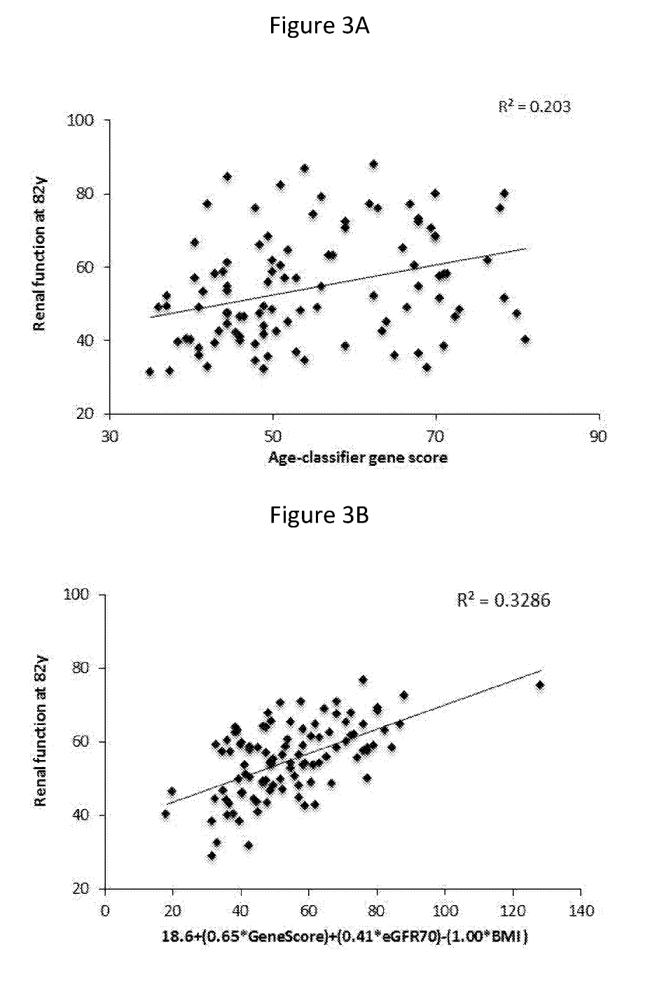

FIG. 3A shows a plot of a cumulative gene score calculated using the 670 genes of Table 1 in ULSAM samples (chronological age=69-70y) against renal function at 82 years.

FIG. 3B shows a multivariate model for prospective renal function at 82 years in the ULSAM cohort.

FIG. 4A shows a Kaplan Meier-plot, with the underlying cox-regression on quartiles of a cumulative gene-score calculated using the 30 genes of Table 4 in ULSAM samples (chronological age=69-70y), with the 3rd and 4th quartiles differing from the^{1st} quartile (p<0.04).

FIG. 4B shows a logistic regression analysis of a cumulative gene-score (continuous variable), calculated using the 30 genes of Table 4 in ULSAM samples (chronological age=69-70y), versus mortality.

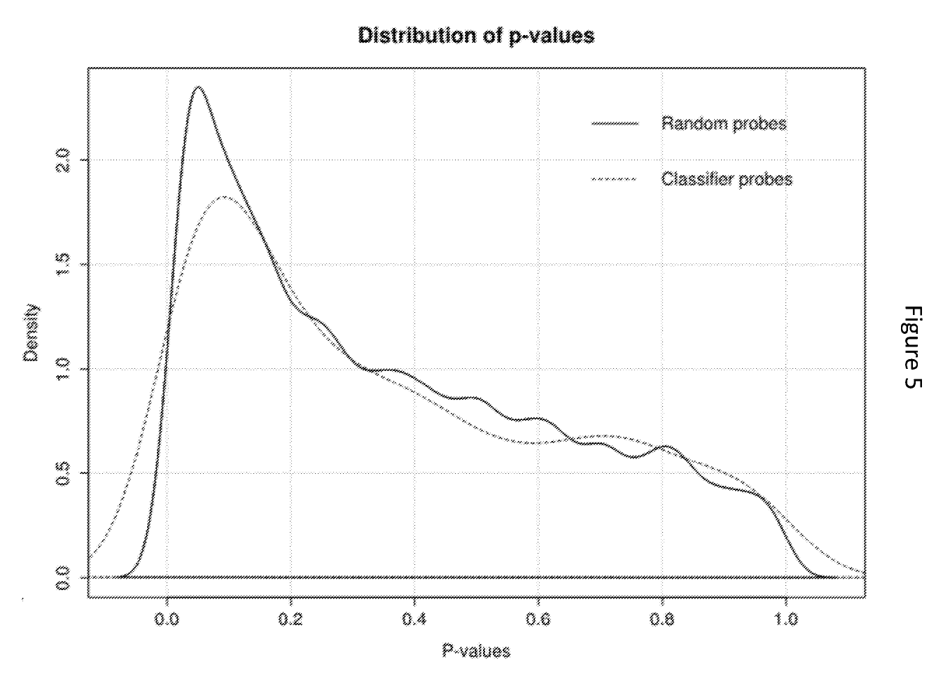

FIG. 5: GO p-value distributions. A plot of the distribution of raw p-values from 10,000 hypergeometric tests using randomly sampled probes (n=670) each time (see solid line) and the distribution of the raw p-values from a hypergeometric test using the 670 probes (classifier probes) associated with the genes of Table 1 (see dotted line).

FIG. 6: A plot showing median gene score in blood (calculated using the 150 genes of Table 2) for patients with AD or MCI vs control samples.

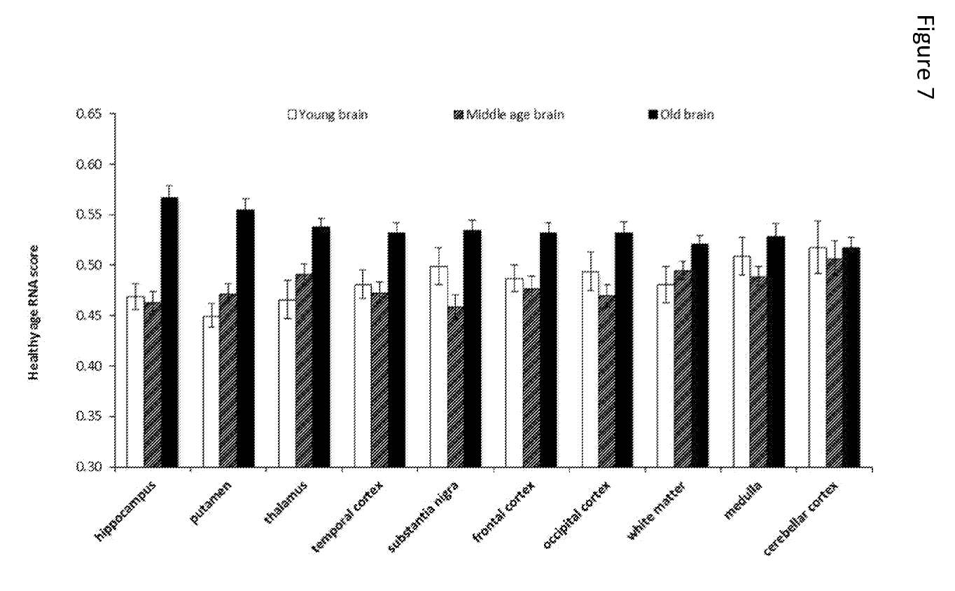

FIG. 7: A graph showing the mean gene score (calculated using the 150 genes of Table 2) for healthy human brain samples from 10 different brain regions with age range across young, middle-aged and old brains.

### ABBREVIATIONS

fRMA Frozen Robust Multi-array Analysis

### GA Genetic Algorithm

GFR Glomerular filtration Rate

### GEO Gene Expression Omnibus

### HOCV Hold Out Cross Validation

### IPA Ingenuity Pathway Analysis

KNN k-Nearest Neighbour

### LOOCV Leave One Out Cross Validation

PGE Positional gene enrichment analysis

### RMA Robust Multi-array Analysis

### ROC Receiver Operating Characteristic

### SNPs Single Nucleotide Polymorphism

### ULSAM Uppsala Longitudinal Study of Adult Men

AD Alzhiemer's disease
MCI Mild Cognitive impairment

### Methods

The following GEO codes represent the source of the raw data used in this project to build and validate the diagnostic/method. STOCKHOLM (GSE59880), DERBY (GSE47881), KRAUS (GSE47969), HOFFMAN (GSE38718), TRAPPE (GSE28422), BRAIN (GSE11882), CAMPBELL (GSE9419), 10 human brain regions (GSE60862), and human skin (Illumina Human HT-12 V3, Arrayexpress: E-TABM-1140). The following GEO codes reflect the clinical validation data sets utilized; ULSAM (GSE48264), and for cognitive health GSE63060 and GSE63061. Informed consent was obtained from all volunteers and ethical approval received from Institutional Research Ethics Committee as reported in primary clinical publications, all studies were conducted under the auspices of the declaration of Helsinki.

For each microarray data set a unique identifier, often defined as a probe or probeset, represents an equivalent section of gene sequence. To go from the microarray technology identifier (the Gene ID in Tables 1-5) to the probeset sequences, gene sequence and the gene name, the probeset identifier is entered into one of several readily available databases, e.g. biomart (http://www.biomart.org) or NetAffx (https://www.affymetrix.com/analysis/index.affx). Alternatively the sequence information from the manufacturer, for each probeset, can be used in BLAST to identify what region of the genome the probeset is complementary too and this also yields identification of the gene name or gene sequence.

### Development, Validation and Optimization of the Healthy/Physiological Age Diagnostics

FIG. 1 provides a schematic overview of the process by which genes detailed in Tables 1-5 were identified. 670 unique probe-sets were identified from a possible starting number of ˜54,000 during step one and these had a variation in classification performance as illustrated. This prototype diagnostic was then developed, evaluating the performance of the entire list, the top-ranked n=150 probe-sets or following an optimization process where a set of n=30 probe-sets were obtained that had improved diagnostic performance when examining a clinical outcome, as illustrated at the end of the work-flow. The process of identification of the probe sets, and the validation of the diagnostic potential of the identified probe sets, is described in more detail below.

The healthy-ageing prototype diagnostic was built using 15 young (<28 year) and 15 older subjects free from metabolic diseases and signs of cardiovascular disease (>59 year): the ‘Stockholm’ data set. Subjects had blood samples taken for glucose measurement and had a fitness test to measure their VO2max. This data allowed us to ensure that the young and older subjects were matched for aerobic fitness, as this parameter has been found to be the most powerful predictor of all cause mortality in humans (Wei et al (1999) Jama 282: 1547-1553; Lee et al (2011), supra). RNA was processed and analysed by Affymetrix gene-chip and the probe-set level intensities of these arrays were normalized using the Robust Multi-array Analysis method (RMA) implemented within the R statistical software environment using the ‘affy’ package (Bioconductor project) (Gentleman et al (2004) Genome Biol 5: R80). When samples are prepared in independent laboratories batch effects are introduced (RNA processing and gene-chip processes, technical variation). To limit these batch effects, the data sets were pre-processed using Frozen Robust Multi-array Analysis (fRMA), adjusting using a robust empirical Bayes framework (Leek et al (2010) Nat Rev Genet 11: 733-739; Leek and Storey J D (2007) PLoS Genet 3: 1724-1735).

The candidate probe-set lists were created via a nested-loop, holding out two arrays at any one time to estimate two parameters from the data. The first of these was the conventional test set result i.e. is the array correctly classified Yes/No. The second novel parameter was used to calculate a rank order for the useful probe-sets. Two-hundred probe-sets were selected during each of the inner-most computational loops by ranking gene expression differences using an empirical Bayesian statistic (implemented as eBayes in the limma′ package) (Smyth (2004) Stat Appl Genet Mol Biol 3: Article 3). All the probe-sets (˜800) involved in the most successful inner-loop iteration were then used as the starting point for the prototype classifier. Probe-sets that targeted multiple genomic loci were then removed from the list and then probe-sets that were involved with a correct identification call 70% of the time or more were carried forward into the rest of the validation process. The model built using the Stockholm data yielded a n=670 probe-set and this is referred to as the prototype healthy-age diagnostic and the specific gene lists are provided in Table 1. An n=150 set was also identified which included probe-sets that were involved in a correct identification call 90% of the time. This set is referred to as the top 150 healthy-age diagnostic and the specific gene lists are provided in Table 2.

Each of the 670 genes was down-regulated in the healthy older subjects compared with the young subjects except for the following genes (which were up-regulated): MED13L, TSPYL1, RBL2, BCKDHB, CUL4A, CAPN1, C6orf62, GNG10, HMGB1, TSC22D1, RAD21, SFRS11, 236978_at, PTP4A2, HNRNPA1, TWF1, PAM, TIA1, JMJD1C, DENND5B, H2AFV, 233674_at, SCP2, INTS6, OGFOD3, PRKAA1, MPDZ, CXorf15, LRRFIP1, TTC17, GPATCH8, BRD2, ASPH, CEP192, 242425_at, RPS6KA5, TTBK2, LATS1, PDE7A, ANK3, 229434_at, SLC11A2, SUZ12, NEAT1, ACSL1, MCL1, NBEA, KANSL1L, TTC3, KRR1, ETNK1, LGI1, PCBP2, 237018_at, FAM76B, FXR1, PRNP, ARMCX3, MBNL1, DERL1, APP, NUCKS1, CFLAR, 239251_at, MYOZ2, SAV1, CEP350, CLIP1, SYNPO2, 242467_at, FUS, WSB1, RBMS3, PPFIBP1, ZNF638, CD47, IFRD1, SFRS18, DHX29, GPAM, PCDH9, 228105_at, 213156_at, B3GNT5, 242457_at, MTMR9, KRIT1, FEZ2, LGR5, NPHP3, MGC24103, PNISR, 229483_at, SKAP2, RUFY3, RP11-271C24.3, 41929_at, MAN2A1, ALDH6A1, LIFR, PFKFB2, ESRRG, TGFBR3, ASH1L, 233073_at, SCAMPI, SRD5A2L2, SKAP2, UNC13C, UNC13C, SPEN, DUFS1, 236439_at, SMCHD1, MALAT1, CD36, MALAT1.

Having identified a prototype set of probe-sets (n=670), classification of independent samples was performed using a k-Nearest Neighbour (KNN, n=3) classifier, implemented in the R ‘class’ package. Leave-One Out Cross Validation (LOOCV) is a specific type of Hold Out Cross Validation (HOCV) which is widely used as a standard procedure to test how well a predictive model is generalized. To implement independent blind validation, we used both independent training and test muscle and brain data sets. That is, we relied on robust external validation methods and not just internal cross validation methods.

To carry out external validation you need two new data sets. In our case the prototype healthy-age diagnostic probe-set list were plotted in multidimensional space, using the Campbell cohort expression values, and this represented the ‘expression space’ of known old and young samples for the subsequent KNN evaluation of subsequent further independent samples e.g. muscle and brain. For the MuTHER cohort skin data-set, which was produced using the Illumina Human HT-12 V3 Bead chip, log-2 transformed signals were normalised per replicate data set, using the quantile normalisation method. A LOOCV approach was used to predict age of all individuals using the 670 genes of Table 1 of the invention or 150 genes of Table 2 of the invention. Genes were mapped to the Illumina platform (551 from 670 genes were represented in this list). For this set of human skin samples, individuals aged < or =45 years were pre-defined as young, and those > or =70 years as old. This was to ensure sufficient numbers of young and old samples existed to fairly assess the classifier performance. Three technical replicates from this skin microarray biobank were analysed separately to establish how reproducible the diagnostic could be in repeated samples from the same clinical sample. Diagnostic performance was judged and optimised using Receiver Operating Characteristic (ROC) analysis (Sing et al (2005) Bioinformatics 21: 3940-3941).

Examples of how refinement of the prototype healthy-age diagnostic set could be achieved was carried out using a Genetic Algorithm (GA) search and an optimisation process was implemented whereby units of probe-sets (e.g. n=30) were randomly selected from the 670 prototype age probe-set list. Each of these of n=30 ‘gene’ units can be conceptually thought of as chromosomes, and a successive number of ‘off-spring’ gene-sets (each of n=30) are created following a cross-over event (Srinivas and Patnaik (1994) Syst Man Cybern IEEE Trans 24: 656-667; Lin et al (2003) J Inf Sci Eng 903: 889-903), analogous to maternal/paternal DNA recombination. Each set of n=30 was also subjected to ‘mutation’ events, where a single probe-set is replaced from a pool of probe-sets from the 670 that were not included in the initial sets of n=30 groupings. The resulting n=30 gene-sets are evaluated on the basis of a fitness function/optimisation criterion which determines if the new population generated is better (e.g. improved ROC performance) than the ‘parent’ gene-sets. Thus, more adaptive chromosomes are kept and less adaptive ones, with lower fitness values, are discarded thereby generating a new population over time. The balance between the rate of the two events, cross-over and mutation, determines the nature of the optimisation process. In contrast to the strategy of the present invention, application of the GA process to exhaustively examine the entire repertoire of probe-sets on the Affymetrix gene-chip (54,000) would be extremely protracted and computationally impossible given the computing resources currently available on earth.

### Production of New Global RNA Profiles for Clinical Validation

Total RNA for the new data sets was extracted from frozen muscle using TRIzol reagent as previously described (Timmons et al (2005) Faseb J 19: 750-760). In vitro transcription (IVT) was performed using the Bioarray high yield RNA transcript labelling kit (P/N 900182, Affymetrix, Inc.). Unincorporated nucleotides from the IVT reaction were removed using the RNeasy column (QIAGEN Inc, USA). Hybridization, washing, staining and scanning of the arrays were performed according to the manufacturer's instructions (Affymetrix, Inc). As a means to control the quality of the individual arrays, all arrays were examined using hierarchical clustering and Normalized Unscaled Standard Error (NUSE, a variance based metric to identify outliers prior to statistical analysis), in addition to the standard quality assessments including scaling factors and chip-housekeeper 573″ratios. The data deposited in GEO that did not originate from our laboratory was also quality assessed. In each case a small number of gene-chips (2-3) were identified that had clear evidence of RNA degradation or other technical defects with the gene-chip profile and these were removed from the analysis.

### ULSAM (Uppsala Longitudinal Study of Adult Men)

This is a cohort of men born in 1920-24 and living in Uppsala, Sweden, who were invited to attend a health examination at the age of 50 years (n=2322) (Dunder et al (2004) Am Heart J 148: 596-601). Re-examinations were performed at 60, 70, 77, 82 and 88 years of age. Over the years the cohort has been very well characterized from metabolic and life-style perspectives. Of specific importance is that the ULSAM subjects were investigated by DEXA scans at both 82 and 88 years of age. Dual-energy X-ray absorptiometry (DEXA) scan measurements were performed during the last decade of the study at these points and yields a measure of loss of lean body mass. Muscle mass status varied between −15% to +10%. from 70 to 88 years old and was unrelated to physical activity scores. Follow-up of these subjects, which included recording their physical activity and exercise status, has been executed at 82 and 88 years of age. Within the subjects are a range of physical activity levels from completely sedentary (˜15%) to recreational-athletic (˜10%). Renal function at age 82 was calculated using cystatin C, which is a marker of GFR (Inker et al (2012), supra). 129 skeletal muscle biopsies were taken from cohort members at 70 years of age in which DEXA and functional testing was performed at 82 and 88 years of age. Skeletal muscle biopsy tissue, taken in 1992, was processed for RNA, extracted with TRizol, in 2012. A total of 108 samples provided good RNA and 50 ng total RNA was amplified using Ambion's WT expression kit to produce cDNA, The cDNA was fragmented and labeled with GeneChip WT Terminal labeling kit (Affymetrix Inc.). The hybridization of cDNA to exon array was 16h at 45 degrees. The arrays were washed in Affymetrix FS450 wash stations and scanned on an Affymetrix 3000 7G scanner according to the manufacturer's instructions. The array data was processed as detailed above.

A gene ranking-based diagnostic methodology was developed and applied to the samples from the ULSAM longitudinal study. The ranking calculation was carried out as follows: for a gene down-regulated with age (in the prototype classifier) subjects were ranked from highest to lowest expression, with the subject with the highest expression assigned 1. For age up-regulated genes the opposite strategy was used. Each subject was then assigned a gene score which was the median of the individual ranking scores for each gene. Regression analysis was used to study the relationship between 70 year age-related gene score and renal function (as renal function is a marker of future mortality in older subjects). In addition to using the gene-score, clinical features of the subjects at 70 years of age were entered into a multivariate model. Model selection was executed using a forwards selection approach, with p>0.1 as stop criterion (backwards selection yielded the same outcome). Variables, previously reported (Dunder et al (2004), supra), were added to the baseline model one at a time, and selected based on p-value (Hagstrom et al (2010) Eur J Heart Fail 12: 1186-1192). For baseline characteristics, and results on univariate analysis see Table 6:

TABLE 6

| Variable | Number of obs. | Mean@70 y | SD | R | R² | P-value |
|---|---|---|---|---|---|---|
| Cystatin C calculated GFR (ml/min) | 123 | 64 | 12 | 0.48 | 0.110 | 0.0006 |
| BMI (kg/m2) | 128 | 25.8 | 2.8 | −1.43 | 0.052 | 0.0172 |
| s-Albumin (g/l) | 126 | 59.9 | 32.1 | −0.12 | 0.045 | 0.0221 |
| Weight (kg) | 128 | 78.9 | 9.9 | −0.37 | 0.042 | 0.0338 |
| OGTT p-gluc 60 min (mmol/l) | 128 | 9.6 | 2.6 | −1.14 | 0.028 | 0.0834 |
| s-Phosphate (mmol/l) | 127 | 43.0 | 2.3 | 1.26 | 0.025 | 0.1036 |
| OGTT p-insulin AUC | 128 | 1.4 | 0.8 | −3.38 | 0.023 | 0.1195 |
| OGTT p-gluc 120 min (mmol/l) | 128 | 7.2 | 2.7 | −0.78 | 0.015 | 0.2164 |
| Free fatty acids (mmol/l) | 128 | 4.0 | 1.0 | 2.14 | 0.014 | 0.2270 |
| OGTT p-gluc 30 min (mmol/l) | 128 | 9.1 | 1.6 | −1.26 | 0.013 | 0.2400 |
| Interleukin-6 (ng/l) | 122 | 3.9 | 4.9 | 0.40 | 0.014 | 0.2432 |
| HDL cholesterol (mmol/l) | 125 | 0.5 | 0.2 | −8.25 | 0.015 | 0.2558 |
| s-Cholesterol ( mmol/l) | 128 | 1.3 | 0.3 | 6.07 | 0.012 | 0.2577 |
| Systolic blood pressure supine (mmHg) | 128 | 145 | 19 | −0.10 | 0.010 | 0.2969 |
| Leisure time physical activity | 125 | 3* | | 2.99 | 0.010 | 0.3221 |
| u-Albumin excretion rate (μg/min) | 122 | 11.8 | 37.1 | −0.05 | 0.009 | 0.3393 |
| s-Triglycerides (mmol/l) | 128 | 6.0 | 1.1 | 1.43 | 0.008 | 0.3648 |
| s-Insulin (pmol/l) | 124 | 45.3 | 20.7 | −0.08 | 0.008 | 0.3673 |
| OGTT p-gluc 0 min (mmol/l) | 128 | 5.5 | 1.0 | 1.20 | 0.004 | 0.5099 |
| Diastolic blood pressure supine (mmHg) | 128 | 84 | 9 | −0.13 | 0.004 | 0.5143 |
| Puls rate (beats/min) | 128 | 65 | 9 | −0.13 | 0.004 | 0.5149 |
| Mini Mental State examination | 121 | 28* | | 0.07 | 0.002 | 0.6276 |
| s-Creatinine (mol/l) | 127 | 340 | 64 | 0.01 | 0.002 | 0.6474 |
| s-Uric acid (mol/l) | 125 | 1.0 | 0.3 | 2.04 | 0.001 | 0.7157 |
| C-reactive protein (mg/l) | 124 | 2.6 | 2.7 | 0.16 | 0.001 | 0.7972 |
| LDL cholesterol (mmol/l) | 126 | 80.2 | 30.8 | 0.01 | 0.0005 | 0.8272 |

Univariate linear regression on baseline characteristics at 70 years of age versus Cystatin C estimated glomerular filtration rate at 82 years of age. Number of obs denotes the number of complete observations available for each variable. Mean and SD denote mean and standard deviation respectively, variables marked with * are categorical and hence reported using median. R denotes the regression-coefficient of the variable. R2 and P-value denote r-squared and p-value of the univariate analysis.

One of the additional candidate variables, BMI, qualified to the final model in those criteria. The final model had the following format: eGFR©82(ml/min)=18.6+0.65*GeneScore+0.41*eGFR70(ml(min)−1.00*BMI (kg/m²)). For the mortality analysis, both the cox-regression and the logistic regression model were implemented in R. For the cox-model the latest ‘survival package’ was used whereas the logistic regression model was estimated using the glm (generalized linear model) function and log it model which models the log odds of the outcome as a linear combination of the predictor variables. Over the observation period, 19 mortality events occurred and the relationship with gene-score was analysed with gene-score as a continuous variable. The exponential regression coefficient for optimised gene-score was 0.93 with a p-value of 0.0002. For the Kaplan-Meier plots, gene-score was divided into quartiles and the plot was produced using the ‘plot-survfit’ function in the survival package. The plot allows overall survival rates to be compared between the four quartiles for gene-score (FIG. 4A). The graph from the logistic regression analysis shows the inverse relationship between the probability of death and gene-score with 95% confidence intervals (FIG. 4B). Both the KM plot and logistic regression plot demonstrate that a better gene-score at the baseline improves the chances of survival and vice-versa.

A prototype multi-gene molecular classifier that could distinguish between healthy young and healthy old tissue samples was produced and validated in ˜600 independent tissue samples. Muscle samples were utilised as a starting point as a large number of independent cohorts were possessed with detailed phenotyping of the donor (Keller et al (2011), supra; Gallagher et al (2010) Genome Med 2: 9). Theoretically, the genes identified should associate with, or reflect, healthy physiological age rather than disease as older subjects were specifically selected that had good aerobic fitness and glucose tolerance (Timmons et al (2010), supra; Gallagher et al (2010), supra). The healthy-age prototype diagnostic was built as previously described, using the following method, with 15 young (˜25 years chronological age) and 15 older subjects (˜65 years chronological age) and this is referred to as the ‘Stockholm’ data.

An ensemble of genes were selected using a Leave-One Out Cross Validation (LOOCV) process where the top 200 probe-sets (RNA detection probes equating to 1 gene) were carried forward during each loop, and each of these probe-sets used to ‘judge’ the age of a second held-out sample, by implementing a k-Nearest Neighbour (KNN, n=3) classifier. Following iterative assessment of all probe-sets on the gene-chip, involving ˜180,000 permutations during which each one of the 30 samples was held-out of the ranking procedure, a repertoire of the best performing ˜800 probe-sets was selected (based on the total number of correct judgements during the 180,000 iterations). The 800 probe-sets were manually inspected and those probe-sets that targeted multiple genomic loci were removed from the classification list, and then probe-sets that were involved with a correct identification call 70% of the time or more were carried forward into the rest of the validation process (FIG. 1). The model built using the Stockholm data yielded n=670 probe-sets and this is referred to as the prototype healthy-age diagnostic and the specific gene lists are provided in Table 1. An n=150 set was also identified which included probe-sets that were involved in a correct identification call 90% of the time. This set is referred to as the top 150 healthy-age diagnostic and the specific gene lists are provided in Table 2. The ‘Stockholm’ data set was discarded from the project at this stage, and a fully independent validation process was carried out, as detailed below.

Prior to undertaking an optimisation process (see below) the ‘raw’ performance of the prototype diagnostic was evaluated, and established if the age of samples obtained could be determined using five independent human muscle cohorts. This was done because an independently validated highly accurate diagnostic of muscle age represents a novel observation in its own right. All the following muscle tissue cohorts were profiled on the same gene-chip platform (Affymetrix U133+2 chip). A new cohort, hereafter named ‘Campbell’, (n=66 chips (Thalacker-Mercer et al (2010) J Nutr Biochem 21: 1076-1082) was used as the new training data-set, used to evaluate the ‘unknown’ independent young and old samples from four additional independent clinical cohorts. This included three existing data-sets from GEO (Trappe′ (Raue et al (2012) J Appl Physiol 112: 1625-1636) (n=48), ‘Hoffman’ (Liu et al (2013) J Gerontol A Biol Sci Med Sci: 1-10) (n=22) and ‘Derby’ (Phillips et al (2013), supra) (n=26)) and a fourth gene-chip dataset (Kraus′, n=33) which was produced from proprietary clinical samples (Slentz et al (2011) Am J Physiol Endocrinol Metab. 301: E1033-9). Remarkably, each clinical sample, from all of these 4 independent clinical cohorts was classified into the correct group, with a success rate of ˜83% (Range 70-93%) for the 670 gene set and ˜93% (Range 70-100%) for the 150 gene set. The 13 gene set (EIF3H, JMJD8, CDK13, TNK2, TNPO2, CALR, CARM1, NRXN2, RAB3A, SIN3A, TFRC, TGFBR3 and U2AF2) yielded success rates of 81% (Derby) and 73% (Trappe). This reproducible result contrasts markedly with methods which study muscle ageing using group mean differential expression analysis (see Phillips et al (2013)). A key feature of the prototype healthy-age diagnostic was that when applied to a group of ‘middle-aged’ subjects with similar chronological age, a highly variable gene-expression score was observed demonstrating that the diagnostic score was distinct from chronological age.

To evaluate if the prototype healthy-age diagnostic reflected age-related changes in other human tissues it was examined if the prototype sets of genes could accurately identify the age of non-muscle human tissues. While it is much less possible to define the ‘health status’ of the non-muscle sources it was felt that the genes, which defined healthy older muscle tissue, should also be modulated to some degree in older versus younger samples, in other tissue types—at least sufficient numbers to provide an accurate ‘fix’ on age—if this was a novel and universal ‘ageing’ signature. Thus, tissue profiles from both ectodermal (brain) and mesodermal (skin) origin were utilised for this purpose. Global RNA profiles from 120 old and young human brain samples (Berchtold et al (2008) Proc Natl Acad Sci USA 105: 15605-15610) were evaluated using the prototype healthy-age diagnostic. The samples represented four brain regions (Entorhinal Cortex (n=25), Hippocampus (n=31), Superior Frontal Gyrus (n=33) and Postcentral Gyrus (n=31)) all of which were certified to be disease-free by histopathology in the original study. The classification success for these human brain samples, using the 670 gene prototype healthy-age diagnostic and muscle gene-chip expression data from a different laboratory as the external independent training set, was an impressive ˜76%. When a brain-tissue expression data-set was used to pre-define the classification space, this success rate improved to ˜84% (see Table 7). Thus, without any refinement, the 670-gene prototype healthy-age diagnostic was also able to distinguish between pathology-free old and young brain samples from independent clinical sources, profiles produced under entirely independent laboratory conditions.

Table 7—Accuracy, Sensitivity and Specificity of the Muscle-Derived Healthy Age Classifier when Applied to Multiple Independent Data Sets.

The sensitivity and specificity of the 670 probe-set derived from the STOCKHOLM gene-chip data was determined for multiple human muscle data sets (Campbell, Derby, Hoffman, Trappe and Kraus) and four brain regions derived from the Berchtold et al (2008) study, supra, with brain set as the training data, and skin from the MuTHER cohort (Glass et al (2013), supra). The majority of data sets demonstrated both high sensitivity and high specificity using the prototype 670 probe-set of Table 1 (shown below in Table 7) or the top-150 prototype list of Table 2. A young sample misclassified as ‘old’ (e.g. in ‘Hoffman’) is noted as a reduced sensitivity. If an old sample was misclassified as being young, as was the case for some of the Hippocampus region, then this is defined as a reduction in specificity, where young is a true-positive in the model. The contributing factors to these misclassifications include lack of standardisation of a single laboratory gene-chip protocol, variation in RNA quality and in some cases older donors that have not induced the ‘healthy ageing’ signature to any extent. The Genetic Algorithm (GA) search and optimisation process was run for 5,000 to 1 million iterations and yielded improved performance, sensitivity and/or specificity in all data sets from only the 670 probe-set as input.

| | Prototype 670 probe-set performance | | | | GA Optimized | | |
|---|---|---|---|---|---|---|---|
| | Sample | Accuracy | | | Accuracy | | |
| Tissue | Size | % | Sensitivity | Specificity | % | Sensitivity | Specificity |
| Muscle (Campbell) | 66 | 82 | 0.83 | 0.80 | — | — | — |
| Muscle (Derby) | 26 | 93 | 1.00 | 0.88 | — | — | — |
| Muscle (Trappe) | 48 | 96 | 0.92 | 1.00 | — | — | — |
| Muscle (Hoffman) | 22 | 73 | 0.79 | 0.63 | >96 | >0.93 | >0.88 |
| Muscle (Kraus) | 33 | 70 | 1.00 | 0.60 | 94 | >0.88 | >0.92 |
| Brain (SFG) | 33 | 88 | 0.86 | 0.89 | — | — | — |
| Brain (PCG) | 31 | 88 | 0.43 | 1.00 | >97 | >0.86 | 1.00 |
| Brain (Hippocampus) | 31 | 81 | 0.33 | 1.00 | 97 | >0.83 | >0.96 |
| Brain (EC) | 25 | 76 | 0.43 | 0.89 | >88 | >0.71 | >0.94 |
| Skin (MuTHER | 279 | 79 | 0.61 | 0.90 | 83-88 | >0.84 | >0.80 |
| Cohort) | | | | | | | |

The prototype healthy-age diagnostic was then used to evaluate the age of human skin samples ((Sawhney et al (2012), supra) and this gene expression data-set originated from a different technology platform: the Illumina Human HT-12 V3 Bead chip. The 670 Affymetrix probe-sets were mapped to gene names, and then to 551 probes on the Illumina chip. There were 279 skin samples for classification analysis, and many of these samples also had two additional technical replicates (n=131 replicate 1; n=124 replicate 2; n=24 replicate 3). The prototype healthy-age classifier gene-list demonstrated good classification success in sets of human skin profiles (79%, see Table 7), confirming that the muscle-derived gene-expression signature appears to be a universal diagnostic of human tissue age and able to operate across technology platforms. This was achieved because of the robust and novel feature selection 2-step process we implemented to build the prototype healthy-age diagnostic and the fact that we uniquely used disease-free older tissue samples.

Assessment of diagnostic performance was achieved using Receiver Operating Characteristic (ROC) analysis ((Sing et al (2005), supra) where both sensitivity and specificity are considered rather than just raw success rates. In fact, the prototype healthy-age signature had excellent sensitivity to specificity ratios in many human clinical cohorts, despite the technical variation and post-mortem processing e.g. brain tissue. However, as access to multiple independent data-sets was possible and promising classification performance was demonstrated, an optimisation process was undertaken to improve ROC performance.

### Optimisation of Age Classifier Performance

Optimisation was undertaken by selecting sub-sets of genes using only the original 670 probe-sets to yield optimal ROC performance for data-sets where sensitivity or specificity could be shown to be further improved (see Table 7). Refinement of the prototype was carried out using a Genetic Algorithm (GA) search and optimisation process was implemented whereby units of probe-sets (e.g. n=30) were randomly selected from the 670 prototype age probe-set list. Each of these of n=30 ‘gene’ units can be conceptually thought of as chromosomes, and a successive number of ‘off-spring’ gene-sets (each of n=30) are created following a cross-over event (Srinivas and Patnaik (1994), supra; Lin et al (2003), supra), analogous to maternal/paternal DNA recombination. Each set of n=30 was also subjected to ‘mutation’ events, where a single probe-set is replaced from a pool of probe-sets from the 670 that were not included in the initial sets of n=30 groupings. The GA process was set to run through a number of recombination events lasting up to 1 million iterations and classifier performance was guided to yield greater specificity or sensitivity depending on which parameter was being improved. This self-adapting process allows the search of the 670 probe-set data to optimise diagnostic performance.

Applying the GA process first to muscle, the ‘Campbell’ data was used as the independent training data-set, and the sensitivity and specificity for n=30 gene-sets to demonstrate improved classification performance of the ‘Hoffman’ and ‘Kraus’ cohorts was determined. For these two cohorts, several n=30 gene-sets were noted which exceeded the prototype performance, where each n=30 probe-set list is largely distinct from each other. For Hoffman, classification success was now 96-100% with near perfect specificity and sensitivity, while a similar result was achieved for the Kraus data set (see Table 7). Similar improvements in performance could be obtained in both brain and skin, such that a number of n=30 gene-sets could be identified using only the original age-classifier prototype gene list that contained sufficient information to determine human tissue age with near perfect success (see Table 7). No single gene was common to all subsets and this is likely to be a key feature of the diagnostic of the invention, as one that successfully operates across numerous diverse tissues and clinical sources should not be driven by a single or small number of biological features.

### Applying the Age Classifier to Determine Long-Term Health in the ULSAM Cohort

The primary hypothesis of the invention was that a validated diagnostic of healthy physiological age could be used to predict health outcomes in a longitudinal study, where subjects were all the same chronological (calendar) age at the point of assessment. When a median rank score was calculated (see below) for twenty middle-aged subjects (Phillips et al (2013), supra), the prototype age-diagnostic gene expression score demonstrated ˜10 times more variation than the chronological age-range, however this in itself does not establish if the information contained within the age signature (the ‘additional’ variance) would be useful for predicting health outcomes. To assess if the prototype healthy-age diagnostic was indeed prognostic, in a longitudinal study, RNA profiles were produced from healthy tissue samples taken and frozen two decades ago from members of the ULSAM cohort (Dunder et al (2004), supra). Each subject was profiled on the Affymetrix EXON 1.0 gene-chip platform and the 670 probe-sets were mapped to the equivalent new probe-sets (yielding 575 probe-sets) so testing the diagnostics ability to work on yet another technology type. The pattern of changes in gene expression between young and healthy old subjects in the prototype age diagnostic was ˜⅔rd down regulated and ˜⅓rd up regulated. Thus, a gene-ranking based diagnostic was calculated taking the direction of gene expression change into account, as described above. The gene-score was, as hoped, unrelated to physical activity levels, the closest surrogate identified herein for physical fitness in the ULSAM cohort so further demonstrating the unique nature of the age diagnostic from conventional clinical tests.

Prior to full optimization (see below) a typical approach to evaluating classification success (Knudsen S (2004) Guide to analysis of DNA microarray data. 2nd ed. Hoboken, N.J.: Wiley-Liss) was taken and used the top 150 healthy-age classifier genes from the prototype list (see Table 2). We generated a cumulative gene-score from the median rank order for all 150 genes for each ULSAM subject. Clinical variables were determined as previously reported (Huang et al (2014) J Intern Med 275(1), 71-83; Zethelius et al (2008) N Engl J Med 358: 2107-2116). Linear regression was used to examine the relationship between the cumulative gene-score of a sample and the respective clinical parameter. As can be observed from plots A-C of FIG. 2 there was no relationship between rank-order for cumulative gene-score and baseline renal function (cystatin-c), blood pressure or total cholesterol (score was unrelated to resting heart rate or physical activity questionnaire scores either). Thus the cumulative gene-score could not be substituted by any of these conventional risk factors (or others listed in Table 6) to predict health-outcomes over the following 20y. Note that at the point of assessment (1992), when the muscle biopsy was taken for subsequent gene-chip profiling, all subjects would be considered in good health for their age and remained physically active.

At 70 years, three subjects had Cystatin C>1.5 mg/I, while by 82 years 36 of the subjects studied in the present analysis had Cystatin C>1.5 mg/L. A 1.5 mg/L Cystatin C corresponds to an estimated GFR of ˜45 mL/min which is borderline for a moderately (30-45 mL/min) elevated risk for all-cause mortality (Zethelius et al (2008), supra). Renal function using Cystatin C was estimated to calculate eGFR, and demonstrated that the baseline healthy-age diagnostic ranking score was related to renal function 12 years later (age 82, p=0.009). An optimized healthy age diagnostic was generated using the GA search and optimisation process (60,000 iterations) yielding an optimised n=30 gene diagnostic (r²=0.203, p<0.000001, Regression Coefficient=0.4504, FIG. 3A and Table 3) for gene-score versus renal function at 82 years. As before, those subjects that ‘switched on’ the healthy-ageing gene expression pattern had superior renal function at age 82 years.

The potential for the healthy-age diagnostic to be combined with clinical variables to provide enhanced prognosis of impaired renal function was investigated using multivariate modeling. In addition to the optimized gene-score, clinical features of the subjects at 70 years of age were considered in the multivariate model. Model selection was executed using a forwards selection approach, with p >0.1 as stop criterion. Variables, previously reported (Dunder et al (2004), supra), were added to the baseline model of gene-score and cystatin C estimated renal function at 70 years of age. A final model utilizing gene-score, eGFR (Estimated Glomerular Filtration Rate) and BMI at a chronological age of 70 years, yielded a model with r²=0.329 (p<0.00001, FIG. 3B). Thus, the gene-score derived from an RNA profile of healthy skeletal muscle (and validated across multiple tissues) was able to combine with two simple clinical measures to capture 33% of the total variance of renal function at 82 years.

The cumulative gene-score was calculated from 670 genes of Table 1 for the USLAM subjects at 70 years of age. While renal function is not sufficiently powerful to predict mortality in disease-free older subjects from the ULSAM cohort (Zethelius et al (2008), supra), it was found that the top 150 healthy age diagnostic was able to predict 20 year survival (p=0.025) in a cox-regression model, with gene-score as a continuous variable.

For those subjects who died during a 20 year follow-up observation period the score was significantly lower than those subjects who remained alive (Wilcoxon test p=0.02). Furthermore, following optimizing of the protoype healthy age diagnostic (GA optimization leading to the 30 genes of Table 4) the baseline gene-score could distinguish between those that had died or not with greater significance (Wilcoxon test p=0.00072).

The GA optimized subset of 30 probes (Table 4) from the prototype (n=670) yielded a strong diagnostic of mortality as demonstrated by logistic regression analysis of gene-score (continuous variable) versus mortality, where the four-fold range in gene-score related to up to a 70% probability of death during the 20 year follow-up period (p=0.00085, FIG. 4B). Further, when dividing this GA optimized gene-score into quartiles, there was a significant difference in survival between the first versus the third and fourth quartiles (p=0.049 and p=0.024) in this cox-regression model (FIG. 4A). Thus, those subjects who died during the observation period started the period with the least induction of the ‘healthy ageing’ expression pattern at chronological age 70 years. The prediction of mortality in the ULSAM 20 year follow-up study is of course preliminary, but it provides further support that induction of the age signature, by the 6^{th} decade of life, represents a positive event since the directional shift in gene-expression and better ‘health’ was consistent for the renal and mortality analysis.

### A Biological Analysis of the Healthy Physiological Age Diagnostic

The RNA signature was evaluated for pathway and gene ontology analysis using both Ingenuity pathway analysis and R-based ontology analysis. There were no significant pathways noted in the Ingenuity analysis, either when using the entire n=670 gene list or when using the sub-set optimised gene lists. While it has previously been demonstrated (Gallagher et al (2010), supra) that applying gene ontology analysis to transcriptome data is problematic due to imprecise knowledge of the true background transcriptome (both tissue specific biases and technology biases mean that certain ontologies can be artificially enriched) it is unusual that a large gene list (n=670 gene), linked to a strong physiological phenotype, is not enriched for specific biological processes. This does however prove that our diagnostic list could not be selected from the literature using prior knowledge.

To confirm this observation, 10,000 random 670 gene-set samples were measured from the entire population of genes measured in the present experiment, and the gene ontology p-value distribution of the random samples was compared with the 670 gene prototype healthy-ageing diagnostic. In FIG. 5 the distribution of raw p-values from 10,000 hypergeometric tests using randomly sampled probes are plotted in black sold lines, while distribution of the raw p-values from a hypergeometric test using the prototype healthy-ageing diagnostic genes are plotted in a dotted line. The analyses clearly demonstrate that the ontological profile of the prototype healthy-ageing diagnostic is not different from a random sample of the starting 54,000 probe-sets, while >98% of the 54,000 probe-sets have no ability to discriminate tissue age.

The inclusion of some previously identified ageing related genes was noted; LMNA (linked with Hutchinson-Gilford Progeria Syndrome), Unc-13 homolog (UNC13C) which is linked with beta-amyloid biology and COL1A1 (thought to change in skin-ageing). It was also examined whether the age-related genes were over represented at genomic loci using Positional enrichment analysis (De Preter et al (2008), supra). The genes from the prototype classifier (the 670 genes claimed herein) found to be over-represented at 7q22 and 11q13. The results were consistent in positional gene enrichment analysis and ToppGene algorithm, both identified 3, 12 and 3 genes at each loci with p<0.001 or less. 11q13 and 11q23 in particular were most significant, and contained genetic variants proven to influence the age of onset of human age-related disease e.g. cancer.

There were in fact a number of significant findings. In particular, 11q13 made a significantly greater contribution (adjusted p-value=0.005-0.007) to the prototype classifier than would be expected by proportionality, while there were a total of 15 genes from the 11q13 and 11q23 over-represented genomic locations (11q13 (ALDH3B1, CAPN1, CDC42EP2, CORO1B, LTBP3, NRXN2, PPP1R14B, RCE1, RCOR2, SART1, SYT12 and ZDHHC24, P=0.0005) and 11q23 (FXYD2, SCN2B and TMPRSS13, P=0.0009)). Interestingly, 11q23 is the location for age-related genetic interactions, namely the apolipoprotein A family (Garasto et al (2003) Ann Hum Genet 67: 54-62; Feitosa et al (2014) Front Genet 5: 159) as well as a region containing genetic association single nucleotide polymorphisms (SNP) which substantially modify for the age of onset of colorectal cancer (Talseth-Palmer et al (2013) Int J Cancer 132: 1556-1564; Lubbe et al (2012) Am J Epidemiol 175: 1-10). Further, 11q13 harbours SNP's associated with age of onset of renal cell carcinoma and prostate cancer and modulating age-related disease emergence by 5yrs (Audenet et al (2014) J Urol 191: 487-492; Lange et al (2012) Prostate 72: 147-156; Jin et al (2012) Hum Genet 131: 1095-1103).

### Healthy Aging Signature and Cognitive Health

A study was carried out of the activation status of the healthy aging signature in blood samples from two large case-control studies of Alzheimer's disease (AD) (publication embargoed GEO data GSE63060 and GSE63061) and it was found that AD patients, and those with early signs of dementia, had a lower median healthy age gene score. The AD cohort has been previously used to study disease pathway changes (Hodges, J. Alzheimers. Dis. 33, 737-53 (2013), Hodges, 30, 685-710 (2012)). 113 subjects aged 75 years or younger in cohort 1 and 112 subjects aged 75 years or younger in cohort 2 were utilised. Using the very oldest subjects in each trial, retrospectively, did not change the outcome of our analysis. Each case-control data-set was ranked for gene-score using only genes selected from the prototype healthy age diagnostic (670 genes, Table 1) and selected from the top 150 healthy age diagnostic (150 genes, Table 2). There is no more than random chance levels of overlap between the healthy aging gene markers, and previously published genomic and genetic disease markers of AD.

AD is a multi-factorial disease (8) with around 22 genetic loci associated with disease risk but no DNA marker is useful in the clinic, as a modifier of risk. Removal of the 7 genes (SKAP2, CEP192, RBM17, NPEPL1, PDLIM7, APP and BIN1) common to the ‘healthy aging gene 670 list’ and previously published genomic markers of AD ((Hodges, J. Alzheimers. Dis. 33, 737-53 (2013), Hodges, 30, 685-710 (2012), Fillit, Alzheimers. Dement. 10, 109-14 (2014); Barmada, Transl. Psychiatry 2, e117 (2012); Amouyel Nat. Genet. 45, 1452-8 (2013); Vellas, J. Alzheimers. Dis. 32, 169-81 (2012); Federoff, Nat. Med. 20, 415-8 (2014) did not alter our results.

Blood RNA from the AD case-control cohort 1 was profiled on Illumina HT-12 V3 bead-chips and Illumina HT-12 V4 for cohort 2. Control subjects were matched in a manner which retained the same chronological age and gender as the AD or MCI subjects. Venous blood for the RNA analysis was collected from the subjects who had fasted 2 hours prior to collection using a PAXgene™ Blood RNA tube (Becton & Dickenson, Qiagene Inc., Valencia, Calif.). The tubes were frozen at −20° C. overnight prior to long-term storage at −80° C. After thawing samples overnight at room temperature, RNA was extracted using PAXgene™ Blood RNA Kit (Qiagen), according to the manufacturer's instructions. The whole genome expression was analyzed using Illumina Human HT-12 v3 Expression BeadChips (Illumina) for the first case-control study and Illumina Human HT-12 v4 Expression BeadChips for the second, independent, case-control study used in our analysis. The expression data was first transformed using variance-stabilization and then quantile normalized using the LUMI package in R. The appropriate probes were mapped from Affymetrix based healthy ageing prototype to Illumina. We calculated a gene-ranking based score in the same manner as for ULSAM data set. Wilcoxon rank sum test from the R stats package was used to test if the median gene score ranks between the two groups, control and AD and control and MCI were significantly different or not.

In cohort 1, the median rank score for AD patients versus chronologically matched controls was highly significantly different (p=0.00089) for 308 genes from the prototype 670 gene list. This confirms the directionality observed for both renal function and mortality in the ULSAM study. Blood RNA from the second AD case-control cohort blood was profiled and in this case 284 genes were common to the prototype 670 gene list. As before, the median rank healthy aging gene-score for AD patients in cohort 2 was significantly lower than the control group (p=0.0099). Furthermore, for both cohort 1 and cohort 2, the median rank healthy ageing gene-score for subjects diagnosed with mild cognitive impairment was lower than that of the chronological age-matched controls (p=0.00000034 and p=0.00055).

When applying the top 150 prototype the probes were mapped from Affymetrix to Illumina yielding 128 genes from the original 150-gene list. The relative median rank score for AD patients was significantly lower than the age and gender matched controls (p=0.004, FIG. 6), based on Wilcoxon rank sum test. Blood RNA from the second AD case-control cohort was profiled on the Illumina HT-12 V4 platform and in this case 122 genes were common to the 150-gene healthy ageing gene score. As before, the median rank healthy ageing gene-score for AD patients in Batch 2 was significantly lower than in the control group (p=0.009, FIG. 6). Furthermore, for both Batch 1 and Batch 2, the median rank healthy aging gene-score for subjects diagnosed with mild cognitive impairment was lower than that of the age-matched controls (MCI, FIG. 6 p=0.00005 and p=0.003 respectively). When applying the 13 gene set (EIF3H, JMJD8, CDK13, TNK2, TNPO2, CALR, CARM1, NRXN2, RAB3A, SIN3A, TFRC, TGFBR3 and U2AF2) the median rank healthy ageing gene-score for AD patients (Batch 1, p=0.043, Batch 2, p=0.051) and MCI patients (Batch 2, p=0.0006) was also significantly lower than in the control group. It is important to note that the control samples used for comparison with MCI overlapped with those used for comparison with AD and that the MCI analysis cannot therefore be considered a fully independent observation. Nevertheless, the greater performance at detecting MCI supports the claim that the age signature in blood can predict disease at least 10 yr in advance.

We also evaluated if the healthy aging signature could act as a diagnostic for AD or MCI when combined with disease biomarkers, and found it exceed current state of the art blood AD diagnostics (when judged using independent data). For example, a combination of a previously published whole blood RNA diagnostic consisting of 48 genes (J. Alzheimer's Disease 33 (2013) 737-753) and the 150-gene healthy aging diagnostic was evaluated using batch 2 samples. The performance of the combined test as a diagnostic for Alzheimer's disease was assessed using a receiver operator characteristic curve yielding an AUC=0.73-0.86. Our healthy aging prototype diagnostic can therefore be combined with disease-specific biomarkers to improve the accuracy of clinical diagnosis or prognosis of age related diseases.

The age diagnostic has allowed the demonstration that patients diagnosed with AD or mild cognitive impairment (many on the cusp of AD), when compared with controls of the same chronological age, had less induction of the healthy aging expression signature in their blood. This diagnostic is the first OMIC signature able to identify AD from controls based entirely on an independently developed research hypothesis that does not include feature selection using disease cohorts.

The induction of the healthy aging expression signature in brain regions with age was also investigated using the BrainEac.org gene-chip resource (GSE60862) which comprises 10 post-mortem brain samples from 134 subjects representing 1,231 samples. Using the 150 genes of Table 2 and same ranking approach as applied to the ULSAM cohort, the median sum of the rank score was calculated for each anatomical brain region (FIG. 7). As before, in healthy older individuals the ‘age’ signature was ‘switched on’ (yielding a greater ranking score). Regulation of the healthy age gene score increased across individual healthy brain regions with chronological age, especially in the hippocampus (p=0.00000002), as well as other regions (putamen, thalamus, substantia nigra and the occipital, frontal and temporal cortex regions (all at least p<0.002 by Holm adjusted Mann-Whitney test). Using the 13 genes (EIF3H, JMJD8, CDK13, TNK2, TNPO2, CALR, CARM1, NRXN2, RAB3A, SIN3A, TFRC, TGFBR3 and U2AF2) the median sum of the rank score increased between young and old brain samples in the hippocampus (p=0.00004).

### DISCUSSION

A change in population age demographics has resulted in an increased prevalence of age-related medical conditions, including cardiovascular and neurodegenerative diseases. It is presumed that successful ageing reflects positive gene-environment interactions that slow the emergence of chronic disease during the 4^{th} to 7^{th} decades of life. Many of the molecular mechanisms which extend the lifespan of laboratory animals have been reported to also positively impact on disease-free lifespan (Kenyon (2010) Nature 464: 504-512). Many of these longevity molecules belong to developmental and growth pathways that impact on important physiological pathways. Nevertheless, it has been difficult to establish if any of these are reliably modulated during human ageing (Phillips et al (2013), supra; Glass et al (2013), supra; Beltran Valls et al (2014) J Gerontol A Biol Sci Med Sci DOI: 10.1093/gerona/glu007). Even if ageing-related molecular mechanisms are conserved across species, such molecules still may not represent reliable clinical biomarkers. In humans, aerobic fitness has been found to be a powerful but limited tiomarker of all-cause mortality (Blair et al (1989), supra; Wei et al (1999) Jama 282: 1547-1553; Myers et al (2002) N Engl J Med 346: 793-801; Church et al (2005) Arch Intern Med 165: 2114-2120), reflecting genetics (Timmons et al (2010), supra), co-morbidity and behavior (e.g. people who feel better may choose to be more physically active). Since the present aim was to develop a RNA diagnostic that when applied to any RNA tissue expression profile, would yield an accurate prediction of healthy physiological age and forecast long-term health, the younger and older samples used in the prototype development were matched for aerobic fitness in an attempt to reveal a novel underlying biomarker.

### Molecular Diagnostics of Human Ageing

Genome-wide association analysis has identified DNA variants associated with human longevity; a trait associated with good long-term health. Sebastinani et al identified 281 DNA variants which collectively explained ˜17% of exceptional longevity in humans (Sebastiani et al (2012), supra) and had a ROC value of only 0.6. Indeed, long-lived humans appear to have a similar genetic burden for common DNA disease variants, suggesting the exceptional longevity model may be the clinical equivalent of the ‘knock-out’ mouse; yielding data that is ultimately difficult to translate to out-bred subjects of ‘normal’ longevity. A recent 27-SNP DNA-based diagnostic (in the Malmo Preventive Project study; 45 year olds) correlated with 23 year blood-pressure increases (Fava et al (2013) Hypertension 61: 319-326). However ROC analysis yielded a poor score of 0.66 (0.5=zero ability) with the established ‘non-genetic’ correlates, and this was not improved using DNA-based data. Thus data with interesting biological association does not always translate into a useful prognostic tool. Thus, while an ageing diagnostic which relies on DNA holds some practical attraction, based on first principles a RNA-based diagnostic is likely to yield superior explanatory power ((Timmons et al (2010), supra).

There have also been several attempts to yield linear models that define the molecular features of chronological age ((Passtoors et al (2012), supra; Phillips et al (2013), supra; Horvath (2013), supra; Hannum et al (2013), supra). In the case of Horvath et al, a methylation based model of chronological age was developed, whereby age was transformed in a unique manner for ages less than and greater than 20 (log and linear transformation respectively). The divergence from chronological age was minimal and thus it is unclear how this can be utilized to identify successful ageing. There was no overlap between the genes in the present healthy-ageing RNA classifier and that of the quasi-linear methylation model derived by Horvath (2013), supra. For the two gene-lists identified by Hannum et al (n=94 and n=326) 4 genes were found to be in common: 1 gene from his primary model (PKM2) and 3 genes from his RNA Methylation association analysis (ANKRD13B, RUNX3 and TCF3) (Hannum et al (2013), supra). It is felt that there will be a fundamental problem with models built on a linear association with chronological age, as such models will not easily distinguish between ‘age’ and the accumulation of molecular features of disease and drug treatment. For this reason, neither RNA nor DNA methylation models, built around linear changes with chronological age, are going to be sufficiently independent of disease variables to be a useful independent diagnostic for predicting long-term health outcomes. In contrast, the present study was able to identify a robust molecular diagnostic of ‘healthy age’ in human tissue, and one that worked in samples of both mesodermal and ectodermal origin.

In a study from Passtoors et al, a set of 21 RNA molecules were reported to ‘mark out’ familial longevity in blood RNA (Passtoors et al (2012), supra) but these correlates had no classification capacity. Further, none of these age-related blood RNA changes replicated in the recent analysis of human brain or muscle (Phillips et al (2013), supra); Glorioso et al (2011) Neurobiol Dis 41: 279-290) indicating that they do not represent a starting point for a multi-tissue diagnostic. It is also true that a novel diagnostic may not supersede chronological age or traditional clinical risk factors for providing prognostic advice. For example, a recent large-scale metabolomic analysis (Fischer et al (2014) PLoS Med 11: e1001606) found that the addition of a significant 4-metabolite signature for mortality did not actually improve risk stratification and the metabolites merely co-varied with age. Strict independent validation is often neglected and in one recent example an RNA diagnostic with excellent ROC performance was reported, but it transpires that the validation data-set used the same control samples as the training-data set invalidating the claim (Ramos et al (2013) Ann Rheum Dis doi: 10.1136/annrheumdis-2013-203405). In fact all published work fails to utilise appropriate independent data to validate their models.

It is perhaps important to explain the primary reasons why it was possible to discover such a robust set of marker genes for healthy physiological age. One major feature of the present research strategy was to build a prototype diagnostic using tissue samples obtained from 65 year subjects who had demonstrated successful ageing i.e. they were selected to have excellent metabolic and cardiovascular health (Keller et al (2011), supra; Gallagher et al (2010), supra). The use of skeletal muscle as a source of high quality RNA for production of a prototype reflects the fact that such material is easily collected from humans (Gallagher et al (2010), supra; Timmons et al (2005), supra) where the functional status of the precise tissue being profiled is readily established. The muscle derived prototype RNA expression pattern was unrelated to several life-style related influences known to impact on muscle phenotype, and the exceptionally high ROC performance in independent muscle, skin and brain tissue profiles, obtained from several countries, demonstrates that a systemic diagnostic of ageing status in humans has been discovered. There was a lack of association between the prototype age diagnostic and various muscle RNA-disease interactions (Keller et al (2011), supra; Fredriksson et al (2008) PLoS One 3: e3686; Stephens et al (2010) Genome Med 2: 1). For example none of the genes modulated in muscle cancer cachexia, wasting or diet-induced muscle atrophy (Thalacker-Mercer et al (2010), supra; Fredriksson et al (2008), supra; Gallagher et al (2012) Clin Cancer Res 18: 2817-2827) appear in the age-diagnostic. Furthermore, the excellent performance in human brain and skin tissue allows us to conclude that it has been possible to identify a robust diagnostic that is not tissue specific and thus is less likely to be related to any tissue-specific environmental interactions or disease processes.

While exceptional longevity (e.g. 100 years or more) is driven by a strong genetic contribution (Sebastiani et al (2012), supra; Puca et al (2001) Proc Natl Acad Sci USA 98: 10505-10508), being fit and healthy at age 65 year is a more common occurrence and likely to reflect complex molecular factors (Kenyon (2010), supra; Sabia et al (2012) CMAJ 184: 1985-1992). The ultimate aim of the invention is to be able to predict long-term health outcomes in middle-aged subjects to facilitate personalization of prevention programs. Ideally, to validate such a new healthy age diagnostic, it would have been desirable to analyze global ‘healthy’ RNA profiles (non-tumorous) from middle-aged subjects with the appropriate 40 year clinical follow-up data. However, no such materials apparently exists. Instead, healthy members of the ULSAM cohort at age 70 years were profiled, and 20 year follow-up data was analysed. In 1992, these 70 year Swedish men were very healthy and physically active for their chronological age, by European or North American standards, while longevity to 90 year of age is not exceptional in the Swedish population (Danielsson and Talback (2012) Scand J Public Health 40: 6-22). The age diagnostic score demonstrated a 4-fold range at 70 years, while chronological age varied by no more than 1 year across the group. Using both the ‘raw’ 670 prototype and the optimised diagnostics, the model of the invention was able to predict health over the following 20 years.

Renal function is an important determinant of all cause mortality (Zethelius et al (2008), supra) and while only 3 from 108 subjects had mild impairment of renal function at 70 years, a clinical model was generated that captured 33% of the variance in renal function at 82 years. The majority of this was driven by the novel healthy-ageing RNA diagnostic of the invention (see FIG. 3B). Despite the small sample size (relative to epidemiological studies) for predicting mortality the fact that the healthy-ageing diagnostic also predicted renal function, is consistent with renal function associating with mortality and morbidity in a number of large epidemiological studies (Zethelius et al (2008) N Engl J Med 358: 2107-2116; Swindell et al (2012) Rejuvenation Res 15: 405-413). The fact that renal function can be diagnosed from a ‘healthy’ muscle RNA profile could be considered remarkable, but the excellent multi-tissue performance of the classifier indicates that the diagnostic should be applicable to any RNA sample, including human blood samples. It is notable that the healthy age diagnostic included genes originating from significantly enriched genomic regions at 11q23 and 11q13 and both regions contain SNPs influencing the age of onset of colorectal, renal and prostate cancer (Garasto et al (2003), supra; Feitosa et al (2014), supra; Talseth-Palmer et al (2013), supra; Lubbe et al (2012), supra; Audenet et al (2014), supra; Lange et al (2012), supra; Jin et al (2012), supra). This is precisely what would be expected if the healthy age diagnostic of the invention was a measure of successful ageing and reflected a set of molecular responses which favoured health in older adults.

### Molecular Features of the Healthy Physiological Age Diagnostic

In a global DNA analysis by Sebastinani et al, the nearest genes to the 281 longevity-related SNPs were related to a number of chronic disease networks (Sebastiani et al (2012), supra), yet in contrast to this link between disease pathways and longevity, long-lived family lines appear to have a similar number of risk alleles for the common age-related chronic diseases (Beekman et al (2010) PNAS 107(42):18046-9). In the present study three genes in the present RNA classifier (erythrocyte membrane protein band 4.1 like 4B (EPB41L4B), calmodulin binding transcription activator 1 (CAMTA1) and the “ageing gene” lamin A/C (LMNA)) relate to three SNPs (rs10512392, rs2032563 and rs915179) from the Sebastinani et al analysis. This provides independent support for two of these previously unvalidated longevity associated genes (EPB41L4B and CAMTA1), while LMNA is a well established component of ageing like disease (Jiang (2013) Nat Med 19: 515). Nevertheless the degree of overlap between these genomic markers of extreme longevity and the present healthy age diagnostic is very limited supporting the idea that these are two distinct phenomena. As noted earlier, the genetic classifier built by Sebastiani et al (2012; supra) yielded an age diagnostic that had a classification sensitivity of 61%, during the validation step, while the present RNA based diagnostic substantially exceeded this performance (>90%). Furthermore, no DNA diagnostic has been shown to capture enough information to be prognostic of long-term health in populations that demonstrate ‘normal’ longevity.

Identification of the molecular processes that contribute to ageing could provide new ideas to tackle age-related functional decline in humans (Curtis et al (2005) Nat Rev Drug Discov 4: 569-580). It has been argued that the natural ageing process reflects a gene-environment interaction whereby genomic variants evolved to enhance early life success impact negatively on health during the transition into older adulthood. The present data suggests that a multi-organ molecular program is induced in those that successfully respond during adulthood and that this process is beneficial. It was noted that a very limited number of young samples have the ‘healthy physiological age’ profile already at 25 years of chronological age (miss-classification equating to reduced sensitivity in Table 7). Whether these are stochastic events or represent true examples of younger subjects with induction of the healthy physiological age profile is unclear. Further, whether induction at an early chronological age reflects a beneficial characteristic or greater exposure to the molecular mediators of ageing would require 40 year longitudinal trials to unravel. For related reasons the majority of ageing mechanisms identified so far have derived from non-primate biological models (Kenyon (2010), supra) and there has been limited ability to validate such mechanisms in humans.

The search for ageing related genes directly in humans has relied on an experimental design that focuses on nonagenarian, centenarians and their siblings or offspring. To this end, differential gene-chip comparisons of human tissue samples (Lu et al (2004) Nature 429: 883-891) and molecular analysis of case-control or cohort studies have been employed to describe some of the gene expression pathways regulated by ageing (Lu et al (2004), supra; McCarroll et al (2004) Nat Genet 36: 197-204). Other strategies for discovering age-related genes such as multi-species RNA expression comparisons, combined with gene ontology analysis, have also been attempted. However, such analysis is compromised by incomplete knowledge of the population of expressed genes utilised as the statistical background for generation of the ontology enrichment scores (Keller et al (2011), supra; Gallagher et al (2010), supra). This renders inter-tissue or inter-species comparisons currently challenging to interpret, as not all genes have an equal probability of appearing in the regulated RNA list. This latter issue relates to both biology (divergence of the molecular characteristics across organisms) and divergent technology (gene-chip performance), factors that no current approach can solve easily.

With these caveats in mind, no significant ontology pathway enrichment was noted within the present 670 prototype (or sub-set) healthy-ageing diagnostic gene lists. In fact, when the ontology profile of the 670 prototype was compared with 10,000 randomly selected 670 gene-sets the distribution of p-values were identical (FIG. 5). The healthy age prototype diagnostic did however demonstrate some linkage with specific genomic regions. The 3 genes from 11q23, also the location for the apolipoprotein A family (Garasto et al (2003), supra; Feitosa et al (2014), supra), originate at a region where single nucleotide variants substantially modify the age of onset of colorectal cancer (Talseth-Palmer et al (2013), supra; Lubbe et al (2012), supra), while at 11q13 several single nucleotide variants modify the age of onset of renal cell carcinoma and prostate cancer (Audenet et al (2014), supra; Lange et al (2012), supra; Jin et al (2012), supra). Thus, while it cannot neatly place the healthy physiological age diagnostic genes into convenient canonical signalling pathways, the technical performance, prediction of human health over 20 years and the association with age-of-onset modifying regions in large human cohort studies, combine to argue that these molecules are genuine markers of human ageing.

In summary, in the present body of work a novel tool has been provided that should enable the future translation of basic science into clinical advances, namely a robust diagnostic of healthy physiological age. A link has been established between induction of the gene expression signature and renal function and mortality in humans over a 20 year follow-up period, which suggests that it may be possible to facilitate healthy ageing in humans through manipulation of the gene-expression networks. The present technology could be used to facilitate the evaluation of anti-ageing related treatment strategies in humans, screen for long-term safety during drug development or augment clinical decision-making that currently inputs chronological age into treatment algorithms.

1. A method of predicting the likelihood of an individual developing an ageing-related disease, or to assist with the diagnosis of an ageing-related disease, which comprises the steps of:
(a) quantifying, in a biological sample from the individual, the level of expression of each of a panel of genes, the panel of genes comprising at least EIF3H, JMJD8, CDK13, TNK2, TNPO2, CALR, CARM1, NRXN2, RAB3A, SIN3A, TFRC, TGFBR3 and U2AF2; and
(b) comparing the level of expression quantified in step (a) with control levels of expression for each of the panel of genes;
such that changes in the levels of expression of the panel of genes are indicative of the individual's risk to developing the ageing-related disease or the presence of the ageing related disease.

2. A method according to claim 1 wherein the panel of genes comprises at least 30, at least 50, at least 70, or at least 120 of the genes listed in Table 2.

3. A method according to claim 1 wherein the panel of genes comprises the 150 genes listed in Table 2.

4. A method according to claim 1 wherein the panel of genes comprises at least 30, at least 50, at least 70, at least 120, or at least 150 of the genes listed in Table 1.

5. A method according to claim 1 in which the biological sample is a blood sample, such as whole blood or blood plasma.

6. A method according to claim 1 in which the biological sample is a tissue sample, such as a tissue sample obtained from the skin, hair, oral mucosa, brain, heart, liver, lungs, stomach, pancreas, kidney, bladder, skeletal muscle, cardiac muscle or smooth muscle.

7. A method according to claim 1 in which the ageing-related disease is Alzheimer's disease, mild cognitive impairment or dementia.

8. A method according to claim 1 in which the ageing-related disease is characterised by a deterioration in renal function.

9. A method of predicting the likelihood of an organ from an individual over >50 years of age being successfully used for transplantation into a donor patient which comprises the steps of:
(a) quantifying, in a biological sample from the individual, the level of expression of each a panel of genes, the panel of genes comprising EIF3H, JMJD8, CDK13, TNK2, TNPO2, CALR, CARM1, NRXN2, RAB3A, SIN3A, TFRC, TGFBR3 and U2AF2; and
(b) comparing the levels of expression quantified in step (a) with control levels of expression for each of the panel of genes;
such that changes in the levels of expression of the panel of genes is indicative of a successful organ transplantation.

10. A method according to claim 9 wherein the panel of genes comprises at least 30, at least 50, at least 70, or at least 120 of the genes listed in Table 2.

11. A method according to claim 9 wherein the panel of genes comprises the 150 genes listed in Table 2.

12. A method according to claim 9 wherein the panel of genes comprises at least 30, at least 50, at least 70, at least 120, or at least 150 of the genes listed in Table 1.

13. A method of assessing the ageing effect of a test compound which comprises the steps of:
(a) incubating the test compound with a biological sample;
(b) quantifying the level of expression of each of a panel of genes, the panel of genes comprising EIF3H, JMJD8, CDK13, TNK2, TNPO2, CALR, CARM1, NRXN2, RAB3A, SIN3A, TFRC, TGFBR3 and U2AF2; and
(c) comparing the levels of expression quantified in step (b), with the levels of expression of each of the panel of genes in the biological sample in the absence of the test compound;
such that a changes in the level of expression is indicative of the ageing effect of the test compound.

14. A method according to claim 13 wherein the panel of genes comprises at least 30, at least 50, at least 70, or at least 120 of the genes listed in Table 2 or comprises the 150 genes listed in Table 2.

15. The method according to claim 13 wherein the panel of genes comprises at least 30, at least 50, at least 70, at least 120, or at least 150 of the genes listed in Table 1

16. Use of a panel of genes comprising at least EIF3H, JMJD8, CDK13, TNK2, TNPO2, CALR, CARM1, NRXN2, RAB3A, SIN3A, TFRC, TGFBR3 and U2AF2 in a method of predicting the likelihood of an individual developing an ageing-related disease, or in a method to assist with the diagnosis of an ageing-related disease, or in a method of predicting the likelihood of an organ from an individual over >50 years of age being successfully used for transplantation into a donor patient.

17. The use according to claim 16 wherein the panel of genes comprises at least 30, at least 50, at least 70, or at least 120 of the genes listed in Table 2 or comprises the 150 genes listed in Table 2.

18. The use according to claim 17 wherein the panel of genes comprises at least 30, at least 50, at least 70, at least 120, or at least 150 of the genes listed in Table 1.
